SEQUENCE LISTING (86 sequences)

SEQ ID NO: 1 - DNA, 34 bp, Artificial Sequence
  Primer
gacccaagcttctgtaagagtagtctcatgatgt

SEQ ID NO: 2 - DNA, 37 bp, Artificial Sequence
  Primer
ttcaggcggccgccaaactgcagtactgttggttcat

SEQ ID NO: 3 - DNA, 34 bp, Artificial Sequence
  Primer
tagaactcgagctgtaagagtagtctcatgatgt

SEQ ID NO: 4 - DNA, 31 bp, Artificial Sequence
  Primer
tagaactcgagtggttactctggacattgat

SEQ ID NO: 5 - DNA, 30 bp, Artificial Sequence
  Primer
ttcagaagcttagggtttccgatggcacct

SEQ ID NO: 6 - DNA, 31 bp, Artificial Sequence
  Primer
tagaactcgagctggatattgcagacactgg

SEQ ID NO: 7 - DNA, 31 bp, Artificial Sequence
  Primer
ttcagggatcctggactagggaggtcctgaa

SEQ ID NO: 8 - DNA, 33 bp, Artificial Sequence
  Primer
tagaagcggccgcaacccaaaaggaaggagcag

SEQ ID NO: 9 - DNA, 34 bp, Artificial Sequence
  Primer
atagaagcggccgcgccatggtgtctcaaccttt

SEQ ID NO: 10 - DNA, 37 bp, Artificial Sequence
  Primer
atagaagtcgacgcaatgttaagtgtcaagaaaaatg

SEQ ID NO: 11 - DNA, 34 bp, Artificial Sequence
  Primer
atagaagcggccgcgtcatcctgtctcccctcct

SEQ ID NO: 12 - DNA, 30 bp, Artificial Sequence
  Primer
tagaagtcgacggtttcctcacaggcaggt

SEQ ID NO: 13 - DNA, 45 bp, Artificial Sequence
  Primer
tagaactcgagacaacaaaatcactagtcttccatctagaatgac

SEQ ID NO: 14 - DNA, 45 bp, Artificial Sequence
  Primer
gtcattctagatggaagactagtgattttgttgtctcgagttcta

SEQ ID NO: 15 - DNA, 29 bp, Artificial Sequence
  Primer
ttcagctcgagggtttccgatggcacctg

SEQ ID NO: 16 - DNA, 32 bp, Artificial Sequence
  Primer
ttcaggcggccgcctggatattgcagacactg

SEQ ID NO: 17 - DNA, 36 bp, Artificial Sequence
  Primer
tagaactcgagcatttcagatcttgatttttagtgt

SEQ ID NO: 18 - DNA, 35 bp, Artificial Sequence
  Primer
gtcatgctagccagtgatgtcagctgttagtaagt

SEQ ID NO: 19 - DNA, 31 bp, Artificial Sequence
  Primer
aattgcggccgccacggaggatagtatgagc

SEQ ID NO: 20 - DNA, 34 bp, Artificial Sequence
  Primer
aatttctagattcattgagacaaaaatcaaatac

SEQ ID NO: 21 - DNA, 18 bp, Artificial Sequence
  Primer
acgtctccagtgtgctga

SEQ ID NO: 22 - DNA, 18 bp, Artificial Sequence
  Primer
cttgacacaggtgccatc

SEQ ID NO: 23 - DNA, 20 bp, Artificial Sequence
  Primer
gtcagccgcatcttcttttg

SEQ ID NO: 24 - DNA, 20 bp, Artificial Sequence
  Primer
gcgcccaatacgaccaaatc

SEQ ID NO: 25 - DNA, 19 bp, Artificial Sequence
  Primer
agttccacgaccagcagct

SEQ ID NO: 26 - DNA, 22 bp, Artificial Sequence
  Primer
cagctgcttgctgatctctgta

SEQ ID NO: 27 - DNA, 18 bp, Artificial Sequence
  Primer
ctgctgctgagaaaacca

SEQ ID NO: 28 - DNA, 18 bp, Artificial Sequence
  Primer
ccttggttttggagccta

SEQ ID NO: 29 - DNA, 18 bp, Artificial Sequence
  Primer
cgagggcaaatacagctt

SEQ ID NO: 30 - DNA, 17 bp, Artificial Sequence
  Primer
gccgtcttcctccatct

SEQ ID NO: 31 - DNA, 18 bp, Artificial Sequence
  Primer
tcttgtcccaccacttga

SEQ ID NO: 32 - DNA, 18 bp, Artificial Sequence
  Primer
cagtgctgagcgtcttct

SEQ ID NO: 33 - DNA, 23 bp, Artificial Sequence
  Primer
acaacaaaatcactagtcttcca

SEQ ID NO: 34 - DNA, 22 bp, Artificial Sequence
  Primer
agctcagaacccagaggtctca

SEQ ID NO: 35 - DNA, 23 bp, Artificial Sequence
  Primer
cggcctgattcacaacaccagct

SEQ ID NO: 36 - DNA, 22 bp, Artificial Sequence
  Primer
tgtaaaccatgatgtgctgcta

SEQ ID NO: 37 - DNA, 60 bp, Artificial Sequence
  Primer
ttggaagacttgaagtcgctggaagacccggagttgttggaagaccttgacacaggtgcc

SEQ ID NO: 38 - DNA, 60 bp, Artificial Sequence
  Primer
ggctgccaataaaagctttgctggtttttggagtacaggtgtgcagctctactccagtct

SEQ ID NO: 39 - DNA, 50 bp, Artificial Sequence
  Primer
tacatggatttgttggaagacatggattttctggaagacatggattttct

SEQ ID NO: 40 - DNA, 28 bp, Artificial Sequence
  Primer
tgggaagactcggatttctgggaagact

SEQ ID NO: 41 - DNA, 23 bp, Artificial Sequence
  Primer
acaacaaaatcactagtcttcca

SEQ ID NO: 42 - DNA, 26 bp, Artificial Sequence
  Primer
cctgcttcaccaccttcttgatgtca

SEQ ID NO: 43 - DNA, 23 bp, Artificial Sequence
  miRNA
agctggtgttgtgaatcaggccg

SEQ ID NO: 44 - DNA, 23 bp, Artificial Sequence
  miRNA
gcctgattcacaacaccagcgcc

SEQ ID NO: 45 - DNA, 23 bp, Artificial Sequence
  miRNA
tggaagactagtgattttgttgt

SEQ ID NO: 46 - DNA, 24 bp, Artificial Sequence
  miRNA
caacaaaatcactagtctacctaa

SEQ ID NO: 47 - DNA, 9035 bp, Artificial Sequence
  vector
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttctgtaagagtagtctcatgatgtcttttaatttaatatctaggccctaatacacaactctttgaatattgctactatctttcagcatgactttccaataccattaagattaagcctttctgttcttcagtttctctaactcaagtcttctaagctccacagcttccaacatctttaattaccagatcttcccaatattataacatccgtgcattccagtccctgtcctgcagcatctccaatgcccttgttttctaaacttcccagtattcaagtcattaaaaatttaggtcttctaatatctccaacaacccatcttcaaagtccaggtcttgtctttcagaatccagagttctaaattacaagtcttccatggtcctaatcctccaatgtctcctatatacaggtcttctagaggctatgtatttcatagtgtttaagtctagattcttcaatgtcttccagcacttacatcattcagtttttacatttttaaatgtctcctactttctagttttacagtgtatttttatcgccaattcctccaatgcccaagtctttaaatacctgcaagaactttcaggtacatcaatatcctcaatgtctgaatgaatagacctcttaatatcctggtctcatattgtccaggctgccagtattcttgacatccagcatctttaaaatttaattcttccaaactgtctttttgacataaatatgtcaatatccatgcttaccaatgtctcccaatatccagggcttctcattttaggatttataaatgtctacatctgctgttgtttatatctgccagtatttaggagttccaaggtacaggacttacaatatctctaatttccagagatttcagtgtctatgacttccaatattcaatatcccaatgttcagggcctccagaaatatcaatgtccagagtaaccaatgtcttcaaaatccaaggcttgcaatgtctccaatatcctgtgcttacaatgtttccaatgtccagggtttccgatggcacctgtgtcaaggtcttccaacaactccgggtcttccagcgacttcaagtcttccaataatctcaaggtcttccagataatcctgagcttccagaaaatccacatcttccagacaatccatgtcttccggacaatccatgtcttccaagaagctccaagtcttccagtaaatcaagtcttccagcaaatccagtcttccagcaattactggtcttccaccaaatccagatcttccaggaaaatccacgtcttccaggaaatccatgtcttccaataatttcaaggtcttccatcaaatacagatcttccagctaatccatgtcttccagaaaaatctgtgtcttccaccaaatccaagtcttccagtaaatctagttcttccagaaaaatctagatcttccagtcaatcagtgtcttccagaaagaaatccaggtcttccagtcaatcagtgtcttccagaaagaaatccaggtcttccagtcagtcagtgtcttccagaaaaatctacgtcttccaccaaatccaggtcttccagtcaatccacatcttccggaaaaaatccaggtcttccagccaatatatgtcttcctgaagatccacgtcttccagaaaatccatgtcttccagaaaatccatgtcttccagtaacctcccagtcttccagaaaatccacgtcttcccaacaatccaagtcttccggataatttgggtcttcctgaaaatctacgtcttccaaaaaagccatgtcttccagaaaatccacatcttccaatggcctccaggtcttccagactatccatgtcttccagaaaatccttgtcttcccttaaatctatagcttccaaaaaatccgggtcttccaggaaatccgtgtcttccagcaagtccacgtcttccaacaaagccatgtcttccagactatccatgtcttccagaaaatccttgtcttccctcaaatccatagcttccgaaaaatccaggtcttccaggaaatccgtgtcttccagcaaatccacgtcttccaacaaagccatgtcttccatcaaattaatgtcttccagcctacttgtgtcttccaacaaaggtacgtcttccaacaaaggtacgtcttccaacaaaggtatgtcttccaacaaaggtacgtcttccagaaaatccacgtcttccaaccaagccatgtcttccagaaaatccacgtcttccagaaaatatatgtcttccaactaagctacgtcttccaacaaatccatgtcttcctatatctccaggtcttccagcatctccagggcttccagcatctgctcgtcttccaacatctccacgtcttccagcatctctgtgtcttccagcatcttcatgtcttccaacaactacccagtcttccatcaactggctcaatatccatgtcttccaacgtctccagtgtgctgatcttctgacattcaggtcttccagtgtctgcaatatccaggtattctaacatgttctatagtccagatcttccagcctttccccaggtccaggccttttcaaatctaggcttcccctgtctgagccttcccatgttaaaatcactctacagaaaaatcttccaactcaactatttctgcttctagttgaattagcttcaaaaacatttaatgggacattcctccttatagaccagatcttatgaaccaacagtactgcagtttggcggccgcttggttactctggacattgatatttctggaggccctgaacattgggatattgaatattggaagtcatagacactgaaatctctggaaattagagatattgtaagtcctgtaccttggaactcctaaatactggcagatataaacaacagcagatgtagacatttataaatcctaaaatgagaagccctggatattgggagacattggtaagcatggatattgacatatttatgtcaaaaagacagtttggaagaattaaattttaaagatgctggatgtcaagaatactggcagcctggacaatatgagaccaggatattaagaggtctattcattcagacattgaggatattgatgtacctgaaagttcttgcaggtatttaaagacttgggcattggaggaattggcgataaaaatacactgtaaaactagaaagtaggagacatttaaaaatgtaaaaactgaatgatgtaagtgctggaagacattgaagaatctagacttaaacactatgaaatacatagcctctagaagacctgtatataggagacattggaggattaggaccatggaagacttgtaatttagaactctggattctgaaagacaagacctggactttgaagatgggttgttggagatattagaagacctaaatttttaatgacttgaatactgggaagtttagaaaacaagggcattggagatgctgcaggacagggactggaatgcacggatgttataatattgggaagatctggtaattaaagatgttggaagctgtggagcttagaagacttgagttagagaaactgaagaacagaaaggcttaatcttaatggtattggaaagtcatgctgaaagatagtagcaatattcaaagagttgtgtattagggcctagatattaaattaaaagacatcatgagactactcttacagcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 48 - DNA, 6857 bp, Artificial Sequence
  vector
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttagggtttccgatggcacctgtgtcaaggtcttccaacaactccgggtcttccagcgacttcaagtcttccaataatctcaaggtcttccagataatcctgagcttccagaaaatccacatcttccagacaatccatgtcttccggacaatccatgtcttccaagaagctccaagtcttccagtaaatcaagtcttccagcaaatccagtcttccagcaattactggtcttccaccaaatccagatcttccaggaaaatccacgtcttccaggaaatccatgtcttccaataatttcaaggtcttccatcaaatacagatcttccagctaatccatgtcttccagaaaaatctgtgtcttccaccaaatccaagtcttccagtaaatctagttcttccagaaaaatctagatcttccagtcaatcagtgtcttccagaaagaaatccaggtcttccagtcaatcagtgtcttccagaaagaaatccaggtcttccagtcagtcagtgtcttccagaaaaatctacgtcttccaccaaatccaggtcttccagtcaatccacatcttccggaaaaaatccaggtcttccagccaatatatgtcttcctgaagatccacgtcttccagaaaatccatgtcttccagaaaatccatgtcttccagtaacctcccagtcttccagaaaatccacgtcttcccaacaatccaagtcttccggataatttgggtcttcctgaaaatctacgtcttccaaaaaagccatgtcttccagaaaatccacatcttccaatggcctccaggtcttccagactatccatgtcttccagaaaatccttgtcttcccttaaatctatagcttccaaaaaatccgggtcttccaggaaatccgtgtcttccagcaagtccacgtcttccaacaaagccatgtcttccagactatccatgtcttccagaaaatccttgtcttccctcaaatccatagcttccgaaaaatccaggtcttccaggaaatccgtgtcttccagcaaatccacgtcttccaacaaagccatgtcttccatcaaattaatgtcttccagcctacttgtgtcttccaacaaaggtacgtcttccaacaaaggtacgtcttccaacaaaggtatgtcttccaacaaaggtacgtcttccagaaaatccacgtcttccaaccaagccatgtcttccagaaaatccacgtcttccagaaaatatatgtcttccaactaagctacgtcttccaacaaatccatgtcttcctatatctccaggtcttccagcatctccagggcttccagcatctgctcgtcttccaacatctccacgtcttccagcatctctgtgtcttccagcatcttcatgtcttccaacaactacccagtcttccatcaactggctcaatatccatgtcttccaacgtctccagtgtgctgatcttctgacattcaggtcttccagtgtctgcaatatccagctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 49 - DNA, 8094 bp, Artificial Sequence
  vector
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttctgtaagagtagtctcatgatgtcttttaatttaatatctaggccctaatacacaactctttgaatattgctactatctttcagcatgactttccaataccattaagattaagcctttctgttcttcagtttctctaactcaagtcttctaagctccacagcttccaacatctttaattaccagatcttcccaatattataacatccgtgcattccagtccctgtcctgcagcatctccaatgcccttgttttctaaacttcccagtattcaagtcattaaaaatttaggtcttctaatatctccaacaacccatcttcaaagtccaggtcttgtctttcagaatccagagttctaaattacaagtcttccatggtcctaatcctccaatgtctcctatatacaggtcttctagaggctatgtatttcatagtgtttaagtctagattcttcaatgtcttccagcacttacatcattcagtttttacatttttaaatgtctcctactttctagttttacagtgtatttttatcgccaattcctccaatgcccaagtctttaaatacctgcaagaactttcaggtacatcaatatcctcaatgtctgaatgaatagacctcttaatatcctggtctcatattgtccaggctgccagtattcttgacatccagcatctttaaaatttaattcttccaaactgtctttttgacataaatatgtcaatatccatgcttaccaatgtctcccaatatccagggcttctcattttaggatttataaatgtctacatctgctgttgtttatatctgccagtatttaggagttccaaggtacaggacttacaatatctctaatttccagagatttcagtgtctatgacttccaatattcaatatcccaatgttcagggcctccagaaatatcaatgtccagagtaaccaatgtcttcaaaatccaaggcttgcaatgtctccaatatcctgtgcttacaatgtttccaatgtccagggtttccgatggcacctgtgtcaaggtcttccaacaactccgggtcttccagcgacttcaagtcttccaataatctcaaggtcttccagataatcctgagcttccagaaaatccacatcttccagacaatccatgtcttccggacaatccatgtcttccaagaagctccaagtcttccagtaaatcaagtcttccagcaaatccagtcttccagcaattactggtcttccaccaaatccagatcttccaggaaaatccacgtcttccaggaaatccatgtcttccaataatttcaaggtcttccatcaaatacagatcttccagctaatccatgtcttccagaaaaatctgtgtcttccaccaaatccaagtcttccagtaaatctagttcttccagaaaaatctagatcttccagtcaatcagtgtcttccagaaagaaatccaggtcttccagtcaatcagtgtcttccagaaagaaatccaggtcttccagtcagtcagtgtcttccagaaaaatctacgtcttccaccaaatccaggtcttccagtcaatccacatcttccggaaaaaatccaggtcttccagccaatatatgtcttcctgaagatccacgtcttccagaaaatccatgtcttccagaaaatccatgtcttccagtaacctcccagtcttccagaaaatccacgtcttcccaacaatccaagtcttccggataatttgggtcttcctgaaaatctacgtcttccaaaaaagccatgtcttccagaaaatccacatcttccaatggcctccaggtcttccagactatccatgtcttccagaaaatccttgtcttcccttaaatctatagcttccaaaaaatccgggtcttccaggaaatccgtgtcttccagcaagtccacgtcttccaacaaagccatgtcttccagactatccatgtcttccagaaaatccttgtcttccctcaaatccatagcttccgaaaaatccaggtcttccaggaaatccgtgtcttccagcaaatccacgtcttccaacaaagccatgtcttccatcaaattaatgtcttccagcctacttgtgtcttccaacaaaggtacgtcttccaacaaaggtacgtcttccaacaaaggtatgtcttccaacaaaggtacgtcttccagaaaatccacgtcttccaaccaagccatgtcttccagaaaatccacgtcttccagaaaatatatgtcttccaactaagctacgtcttccaacaaatccatgtcttcctatatctccaggtcttccagcatctccagggcttccagcatctgctcgtcttccaacatctccacgtcttccagcatctctgtgtcttccagcatcttcatgtcttccaacaactacccagtcttccatcaactggctcaatatccatgtcttccaacgtctccagtgtgctgatcttctgacattcaggtcttccagtgtctgcaatatccaggtattctaacatgttctatagtccagatcttccagcctttccccaggtccaggccttttcaaatctaggcttcccctgtctgagccttcccatgttaaaatcactctacagaaaaatcttccaactcaactatttctgcttctagttgaattagcttcaaaaacatttaatgggacattcctccttatagaccagatcttatgaaccaacagtactgcagtttggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 50 - DNA, 7924 bp, Artificial Sequence
  vector
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatcctggactagggaggtcctgaaggaggagggataaatattttcttacacacgttaaatattaaaatctttgttacaagcacattttggtcagtggcttttagctcttacactttaagttttgacttctgtatctgtctgtctctgtctgtctctgtctgtctgtctgtctttgtctgtctgtctgtttctctttgcatcacatctctgtctttttggttgcaatcataattcttccattatgttcagcatttcagatcttgatttttagtgttcagccctacagccacatgatatcttaaactctgaagaagagacaagttttgggactggtgacaattgtctagagagcatggagggccatgtcaagcgccccatgaatgcatttatggtgtggtcccgtggtgagaggcacaagttggcccagcagaatcccagcatgcaaaatacagagatcagcaagcagctgggatgcaggtggaaaagccttacagaagccgaaaaaaggccctttttccaggaggcacagagattgaagatcctacacagagagaaatacccaaactataaatatcagcctcatcggagggctaaagtgtcacagaggagtggcattttacagcctgcagttgcctcaacaaaactgtacaaccttctgcagtgggacaggaacccacatgccatcacatacaggcaagactggagtagagctgcacacctgtactccaaaaaccagcaaagcttttattggcagcctgttgatatccccactgggcacctgcagcagcagcagcagcagcagcagcagcagcagttccataaccaccaccagcagcaacagcagttctatgaccaccaccagcagcagcagcagcagcagcagcagcagcagcagttccatgaccaccaccagcagaagcagcagtttcatgaccaccaccagcagcaacagcagttccatgaccaccaccaccaccaccaggagcagcagttccatgaccaccaccagcagcaacagcagttccatgaccaccagcagcagcagcagcagcagcagcagcagcagttccatgaccaccaccagcagaagcagcagttccatgaccaccaccaccaccaacagcagcagcagttccatgaccaccagcagcagcagcagcagttccatgaccaccagcagcagcagcatcagttccatgaccacccccagcagaagcagcagttccatgaccacccccagcagcaacagcagttccatgaccaccaccaccagcagcagcagaagcagcagttccatgaccaccaccagcagaagcagcagttccatgaccaccaccagcagaagcagcagttccatgaccaccaccagcagcaacagcagttccatgaccaccaccagcagcagcagcagcagcagcagcagcagcagcagcagttccacgaccagcagcttacctacttactaacagctgacatcactggtgagcatacaccataccaggagcacctcagcacagccctgtggttggcagtctcatgacactggccttttctcctacctatgccaacactcccccttgctatgatttttaagtctgagattcctagttggtttagatactgacttttctcctgtcatgaaaaaggtcctgctccttccttttgggttgcggccgctcgacgatctctatcactgatagggagatctctatcactgatagggagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccataggggatcgggagatctcccgatccgtcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 51 - RNA, 22 nt, Homo sapiens
uggaagacuagugauuuuguug

SEQ ID NO: 52 - RNA, 22 nt, Homo sapiens
caacaaaucacagucugccaua

SEQ ID NO: 53 - RNA, 22 nt, Homo sapiens
caacaaaucccagucuaccuaa

SEQ ID NO: 54 - RNA, 22 nt, Homo sapiens
uagcuuaucagacugauguuga

SEQ ID NO: 55 - RNA, 21 nt, Homo sapiens
caacaccagucgaugggcugu

SEQ ID NO: 56 - RNA, 23 nt, Homo sapiens
uuuggcacuagcacauuuuugcu

SEQ ID NO: 57 - RNA, 22 nt, Homo sapiens
aaucaugugcagugccaauaug

SEQ ID NO: 58 - RNA, 22 nt, Homo sapiens
uggagagaaaggcaguuccuga

SEQ ID NO: 59 - RNA, 22 nt, Homo sapiens
aggggcuggcuuuccucugguc

SEQ ID NO: 60 - RNA, 23 nt, Homo sapiens
cgcauccccuagggcauuggugu

SEQ ID NO: 61 - RNA, 20 nt, Homo sapiens
acugccccaggugcugcugg

SEQ ID NO: 62 - RNA, 22 nt, Homo sapiens
uuugguccccuucaaccagcua

SEQ ID NO: 63 - RNA, 22 nt, Homo sapiens
uccuucauuccaccggagucug

SEQ ID NO: 64 - RNA, 21 nt, Homo sapiens
gauuucaguggagugaaguuc

SEQ ID NO: 65 - RNA, 22 nt, Homo sapiens
caucuuaccggacagugcugga

SEQ ID NO: 66 - RNA, 22 nt, Homo sapiens
uaacacugucugguaacgaugu

SEQ ID NO: 67 - RNA, 22 nt, Homo sapiens
caucuuacugggcagcauugga

SEQ ID NO: 68 - RNA, 22 nt, Homo sapiens
uaauacugccugguaaugauga

SEQ ID NO: 69 - RNA, 22 nt, Homo sapiens
cgucuuacccagcaguguuugg

SEQ ID NO: 70 - RNA, 23 nt, Homo sapiens
uaauacugccggguaaugaugga

SEQ ID NO: 71 - RNA, 22 nt, Homo sapiens
caucuuccaguacaguguugga

SEQ ID NO: 72 - RNA, 22 nt, Homo sapiens
uaacacugucugguaaagaugg

SEQ ID NO: 73 - RNA, 22 nt, Homo sapiens
uaauacugucugguaaaaccgu

SEQ ID NO: 74 - RNA, 22 nt, Homo sapiens
accguggcuuucgauuguuacu

SEQ ID NO: 75 - RNA, 22 nt, Homo sapiens
uaacagucuacagccauggucg

SEQ ID NO: 76 - RNA, 22 nt, Homo sapiens
ugugacugguugaccagagggg

SEQ ID NO: 77 - RNA, 22 nt, Homo sapiens
cguguucacagcggaccuugau

SEQ ID NO: 78 - RNA, 20 nt, Homo sapiens
uaaggcacgcggugaaugcc

SEQ ID NO: 79 - RNA, 23 nt, Homo sapiens
uuaaugcuaaucgugauaggggu

SEQ ID NO: 80 - RNA, 22 nt, Homo sapiens
cuccuacauauuagcauuaaca

SEQ ID NO: 81 - RNA, 22 nt, Homo sapiens
accuggcauacaauguagauuu

SEQ ID NO: 82 - RNA, 23 nt, Homo sapiens
agcuacauugucugcuggguuuc

SEQ ID NO: 83 - RNA, 22 nt, Homo sapiens
cucaguagccaguguagauccu

SEQ ID NO: 84 - RNA, 21 nt, Homo sapiens
agcuacaucuggcuacugggu

SEQ ID NO: 85 - RNA, 22 nt, Homo sapiens
uggagugugacaaugguguuug

SEQ ID NO: 86 - RNA, 22 nt, Homo sapiens
aacgccauuaucacacuaaaua

(19) United States
(12) Patent Application Publication
(10) Pub. No.: US 2015/0299702 A1
(43) Pub. Date: Oct. 22, 2015

(54) CIRCULAR RNA FOR INHIBITION OF MICRORNA

(71) Applicants: Peter Samuel WULFF, Frederiksberg (DK); Mogens TERGÅRD DUCH, Risskov (DK); Shervin BAHRAMI, Aarhus C (DK); Jørgen KJEMS, Risskov (DK); Hansen Thomas BIRKBALLE, Aarhus C (DK)

(72) Inventors: Jørgen Kjems, Risskov (DK); Hansen Thomas Birkballe, Aarhus C (DK)

(73) Assignee: Aarhus Universitet, Arhus C (DK)

(21) Appl. No.: 14/648,360

(22) PCT Filed: Nov. 29, 2013
(86) PCT No.: PCT/DK2013/050404

§ 371 Date: May 29, 2015

Related U.S. Application Data
(60) Provisional application No. 61/731,788, filed on Nov. 30, 2012.

(51) Int. Cl. C12N 15/113

(52) U.S. Cl. CPC C12N 15/113; C12N 2310/113; C12N 2310/532; C12N 2320/30

(57) ABSTRACT

The present invention relates to inhibition of microRNAs (miRNA), other types of RNA and RNA-binding proteins. In particular the present invention relates to nucleic acid sequences that are circular and allow inhibition of the interactions between microRNAs and their RNA target and being resistant to degradation by exonucleases. Also, the present invention relates to nucleic acid sequence that are circular and acts as an RNA blocker or protein decoy.

### TECHNICAL FIELD OF THE INVENTION

The present invention relates to inhibition of microRNAs (miRNA), other types of RNA and RNA-binding proteins. In particular the present invention relates to nucleic acid sequences that are circular and allow inhibition of the interactions between microRNAs and their RNA target. Also, the present invention relates to nucleic acid sequences that are circular and acts as an RNA blocker or protein decoy.

### BACKGROUND OF THE INVENTION

MicroRNAs (miRNA) are small important regulators of gene expression and are currently believed to regulate approximately 70% of human genes.

More than a thousand different miRNA have been characterized in the human genome and they all are assumed to function by a similar mechanism: The miRNAs base-pair with target messenger RNA (mRNA) and recruit nucleases that degrade the targeted RNA from the termini and/or inhibit translation.

In cancer and many other diseases, deregulation of gene-expression is observed and in many cases miRNAs have been shown to play an integral part or even the causative role in disease development.

Therefore, inhibiting miRNA activity is a promising approach not only in potential treatment of the disease but also in achieving a basic understanding of miRNA functions.

Hansen et al (EMBO J, 2011, 30, 4414-22) is a publication by the present inventors in which the function of miR-671 and a circular antisense transcript of CDR1 is examined.

The circular transcript is shown to be cleaved by miR-671, an unusual mechanism for a human miRNA, but no clear function or mechanism is identified.

WO 2010/084371 is a patent application that discloses circular interfering RNA molecules (ciRNA) which comprise sense and antisense strands connected by a loop i.e. they are double stranded.

To date, miRNA inhibition is usually performed with linear synthetic and chemically modified so-called anti-miRs.

These anti-miRs, when introduced into cells in high concentration, attach itself to the miRNA and inactivate it.

Disadvantages of these anti-miRs are the high concentration which leads to a higher risk of off-target effects, the problem of delivery of the anti-miRs in a sufficiently high concentration inside the cells and furthermore the linear nature of these anti-miRs allow degradation by exonucleases.

Inhibitors of the specific interactions between microRNAs and their mRNA target, which are produced as a natural RNA by transcription inside the cells, but resistant to degradation by exonucleases, are desired.

### SUMMARY OF THE INVENTION

Thus, an aspect of the present invention relates to a RNA polynucleotide which is circular, which is capable of inhibiting the specific interaction between microRNAs and their mRNA target while being resistant to degradation by exonucleases.

One aspect of the invention relates to a circular RNA which is at least partly complementary to a human microRNA sequence.

Another aspect of the present invention relates to a RNA polynucleotide comprising a splice acceptor site (SA) upstream of one or more copies of nucleic acid sequence that is partially complementary to a human microRNA sequence, and a splice donor site (SD) downstream of the nucleic acid sequence that is partially complementary to a human microRNA sequence.

Yet another aspect of the present invention is to provide a polynucleotide including but not limited to a deoxyribonucleic acid (DNA) vector comprising a promoter and sequence encoding the RNA capable of circularization and which contains one or more nucleic acid sequences that are a guide sequence complementary to the seed sequence of a human microRNA sequence. Circularization of the transcript can be achieved by, but not limited to, providing a splice acceptor (SA) site upstream of a splice donor (SD) site in addition to an inverted repeat. Yet another aspect of the present invention is a DNA containing an inverted repeat partially or fully outside the RNA encoding segment to direct the circularization. Yet another aspect of the present invention is to express the RNA from a plasmid, virus or from a stably integrated DNA segment in the genome of the cell. Yet another aspect of the present invention is to obtain the circularization by using engineered RNA self-splicing or by enzymatic or chemical ligation in vitro prior to introduction to cells. Yet another aspect of the present invention is to use other RNA ligase activities in the cell to form circles including tRNA ligase. Yet another aspect of the present invention is to use stable intron lariats as RNA circles.

In yet another aspect, the invention comprises a splice acceptor site (SA) upstream of a nucleic acid sequence that is a guide sequence corresponding to the seed sequence of a human microRNA sequence, and a splice donor site (SD) downstream of the nucleic acid sequence that is a guide sequence complementary to the seed sequence of a human microRNA sequence.

Further aspects of the present invention relates to a cell comprising these polynucleotides and pharmaceutical compositions comprising the circular polynucleotide RNA of the present invention and a pharmaceutically acceptable diluent, carrier, or adjuvant.

Yet other aspects of the present invention relates to a method for inactivating the function of microRNA or reducing the effective amount of a microRNA in a cell or an organism or a method for de-repression of a microRNA targeted mRNA in a cell or an organism, comprising administering a pharmaceutical composition of the present invention, to the cell or the organism.

Another aspect of the present invention relates to the use of a polynucleotide circular RNA as of the present invention for the manufacture of a medicament for the treatment of a disease or medical disorder associated with the presence or over-expression of the microRNA.

A further aspect of the present invention relates to a method for the treatment of a disease or medical disorder associated with the presence or over-expression of the microRNA, comprising the step of administering a pharmaceutical composition of the present invention to a person in need thereof.

### BRIEF DESCRIPTION OF THE FIGURES

FIG. 1

CiRS-7: a, b, Schematic representation of the genomic locus encoding the human (a) and mouse (b) CiRS-7 and predicted miR-7 and miR-671 target sites (greyscaled according to target site strength, see legend) are shown. Vertebrate conservation scores are depicted and the horizontal black bar represents the genomic location of CiRS-7. In a, zoom-in of a CiRS-7 subregion is shown below. In b, Agog HITS-CLIP reads mapped onto CiRS-7 locus are shown. c, CiRS-7 expression vectors; CiRS-7-fs consists of the CiRS-7-exon without flanking sequences and splice sites. CiRS-7-ir includes splice sites and the endogenous flanking genomic sequence (1000 bp upstream and 200 bp downstream). In CiRS-7, a part of the upstream sequence has been inverted and inserted downstream, as illustrated by the directional bar. d, Northern blot with 5 ug of RNA from HEK293 cells transfected with EV (pcDNA3), CiRS-7-ir (pcDNA3-CiR7-ir), CiRS-7 (pcDNA3-CiRS-7) or total RNA from human brain. The blot was probed against CiRS-7 and 18S (loading control). Migration of circular CiRS-7 and vector-based non-circular product (*) is marked. e, Northern blot with 10 ug (agarose northern, upper two panels) or 20 ug (PAGE northern, lower two panels) using RNA from HEK293 cells co-transfected with EV, CiRS-7-fs or CiRS-7 together with miR-7 or miR-769 expression vectors (pJEBB-7 or pJEBB-769, respectively). The blots were hybridized with probes as indicated next to each panel. 18S and miR-15b serves as loading controls.

FIG. 2

Interaction between CiRS-7 and miR-7: a, Immunoprecipitation (IP) of myc-tagged Agog from HEK293 cells transfected with either myc-Ago2 and pJEBB-7 or myc-Ago2 and pJEBB-769. Empty vector (EV) serves as myc-Ago2 negative control. CiRS-7 and GAPDH levels are quantified by RT-qPCR and the relative IP/Input ratios are plotted. b, 3′ end biotinylated miRNA duplexes were transfected into HEK293 cells. After streptavidin capture, the input and bound fractions were evaluated by RT-qPCR (analysed as in a). c, RNA FISH on HEK293 cells co-transfected with pcDNA3-CiRS-7 and the pCK-7. CiRS-7 and miR-7 probes were labelled with Cy5 and Cy3, respectively. d, IF-FISH on CiRS-7-transfected HEK293 cells with or without miR-7 (pCK-7) co-transfection immunoblotted with hDcp1A antibody and hybridized with Cy5-labelled CiRS-7 probe. e, f, In situ hybridization of CiRS-7 showing intense neuronal expression throughout neocortex (NCx), hippocampal subregions CA1 and CA3, and dentate gyrus (DG). Arrows in f point at CiRS-7 expressing interneurons situated at the transition between stratum radiatum (r) and the molecular layer (m) of CA1. g, h, In situ hybridization showing miR-7 expression in neurons and glial cells. Arrows in h point at miR-7 expressing interneurons. miR-7 expressing glial cells are numerous in the corpus callosum (cc) where they are organized in rows between the myelinated fibre bundles. Small miR-7 expressing glial cells are also widely distributed throughout the hippocampal neuropil layers. g, granule cell layer; h, dentate hilus; o, stratum oriens CA1; r, stratum radiatum CA1. Bars: 200 μm (e, g) and 100 μm (f, h).

FIG. 3

CiRS-7 sponges miR-7 activity: a, b, Luciferase reporter assays using reporter constructs with either a perfect target site (a) or the entire CiRS-7 sequence (b) inserted into the 3′UTR of renilla luciferase. Forty eight hours after transfection with luciferase expression vectors (psiCheck) and the indicated constructs (in case of anti-miR, a final concentration of 50 nM was used), luminescence was measured. The relative levels of renilla luminescence, normalized to firefly, are plotted. c, e, f, g, RT-qPCR quantification of CiRS-7 (c), SNCA (d), EGFR (e) and IRS2 (f) levels in HeLa-CiRS-7 or HeLa-EV cells transfected with gradient concentration of miR-7 as indicated. g, h, I, j, RT-qPCR quantification of CiRS-7 (g), SNCA (h), EGFR (i) and IRS2 (j) levels in HeLa-CiRS-7 cells transfected with miR-671 or N.C. (negative control) 24 hrs prior to transfections with miR-7 gradient. (*, p<0.05; **, p<0.01)

FIG. 4

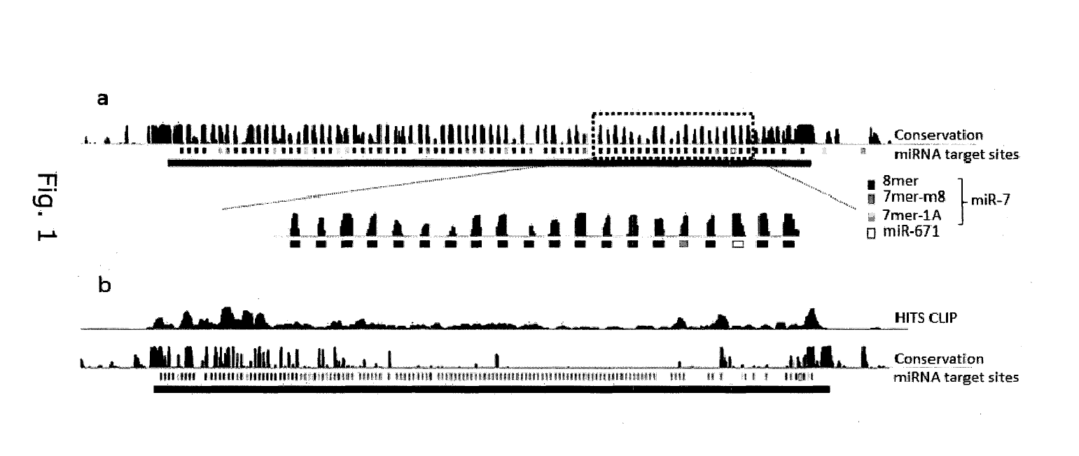

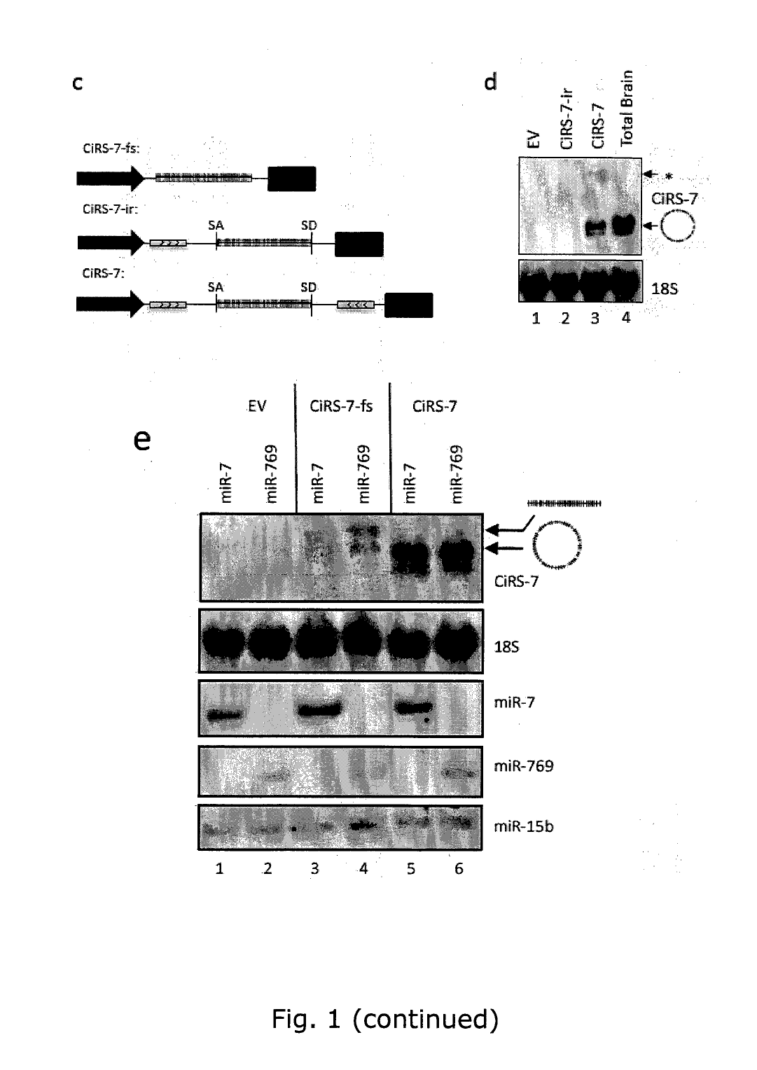

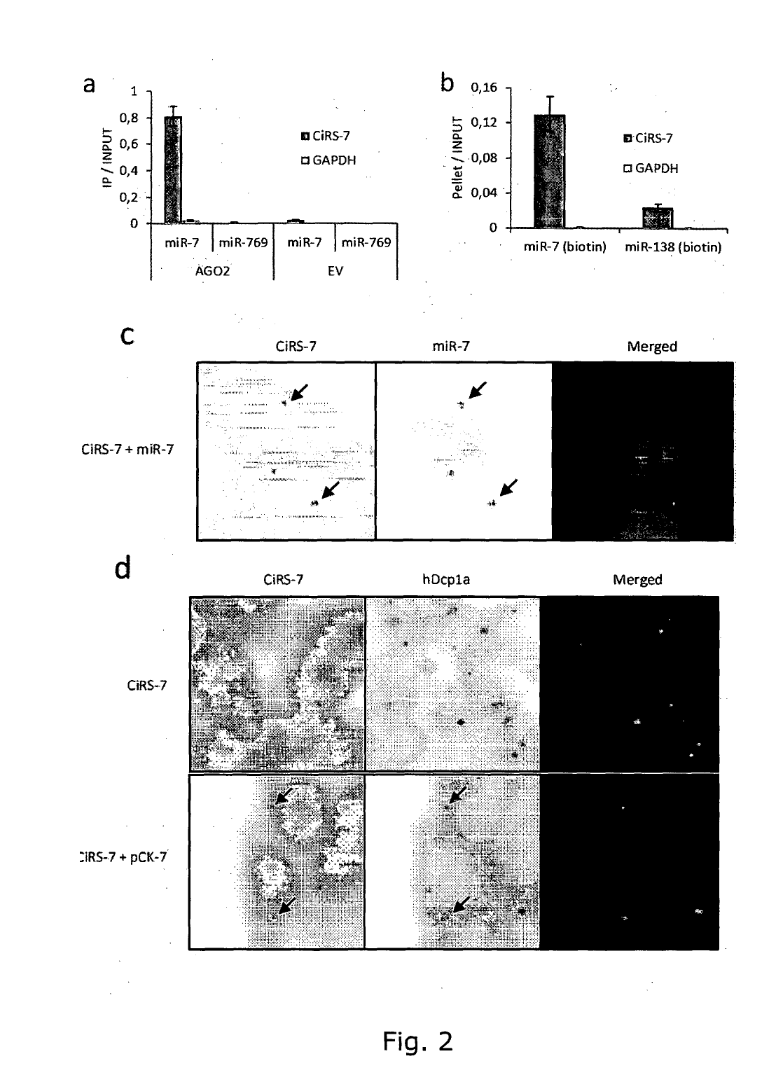

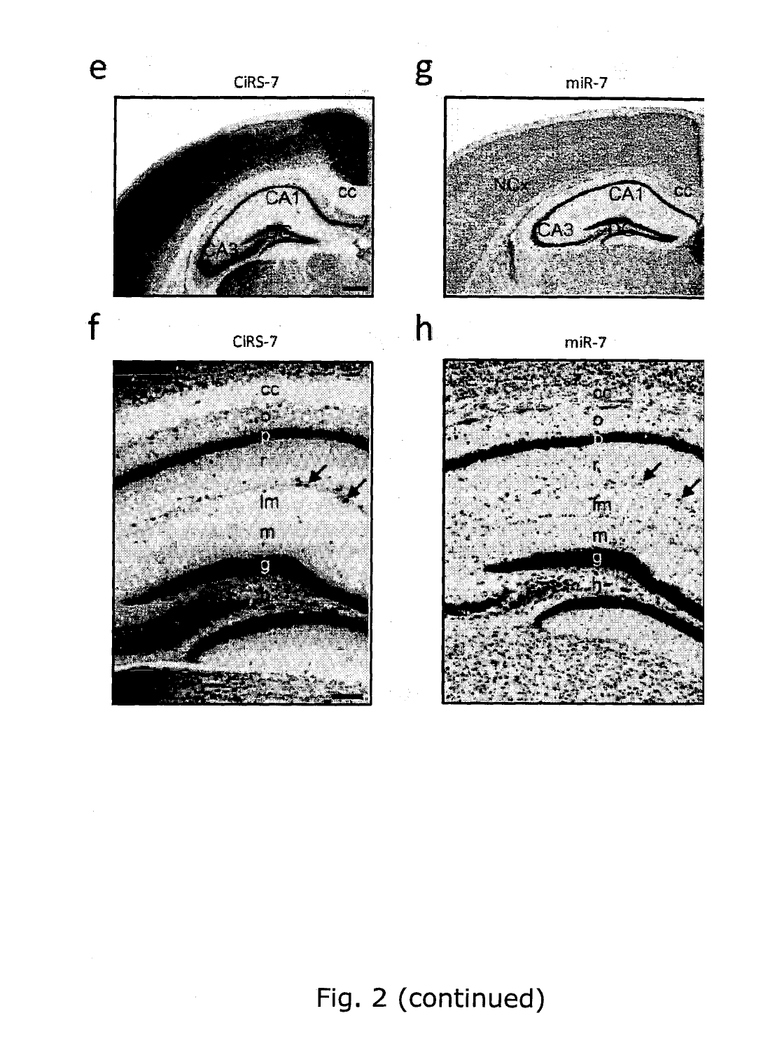

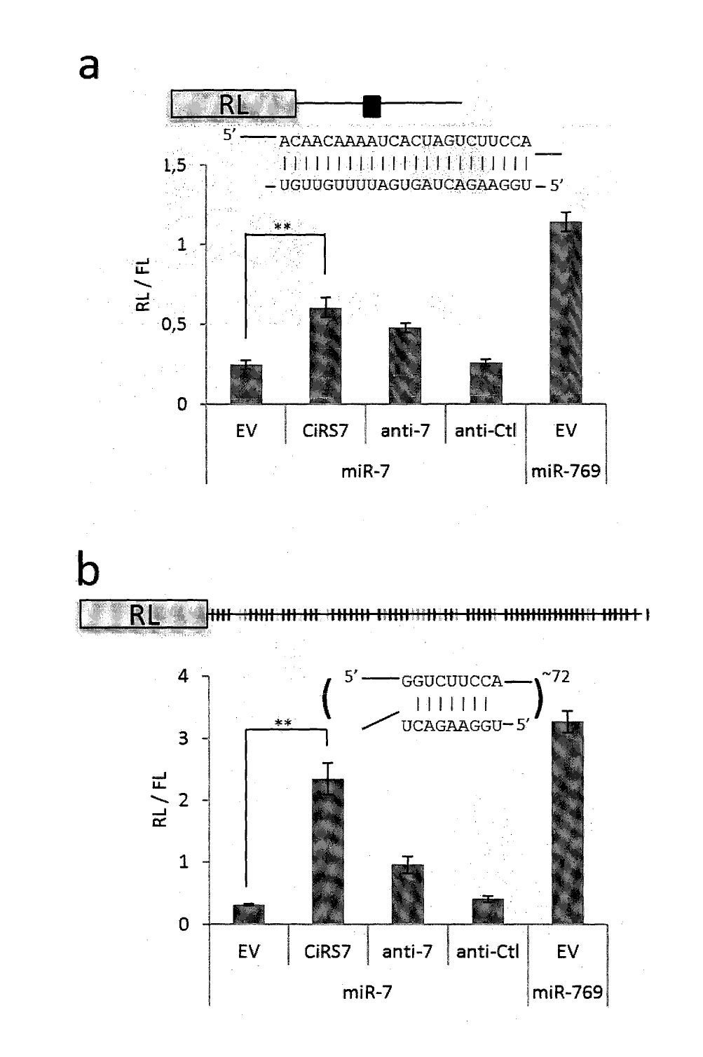

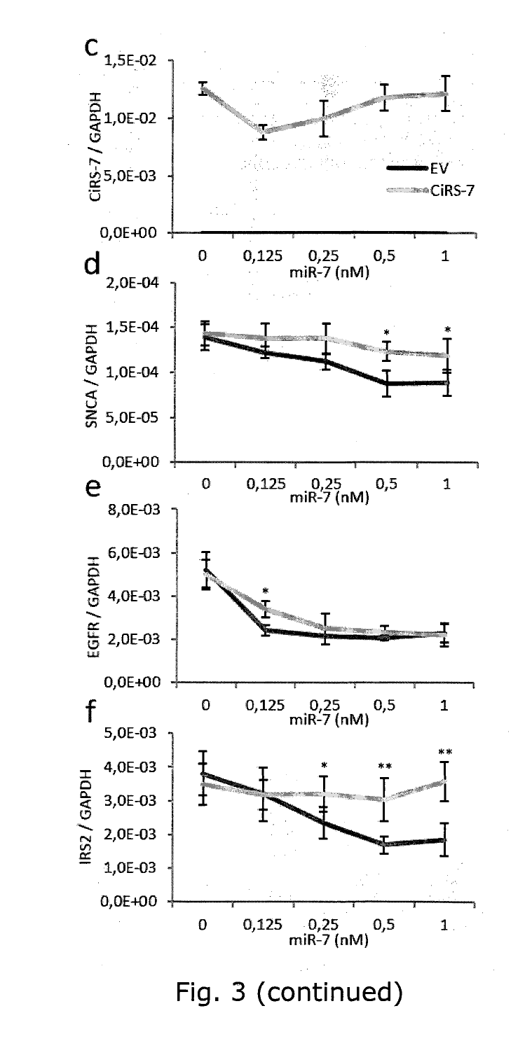

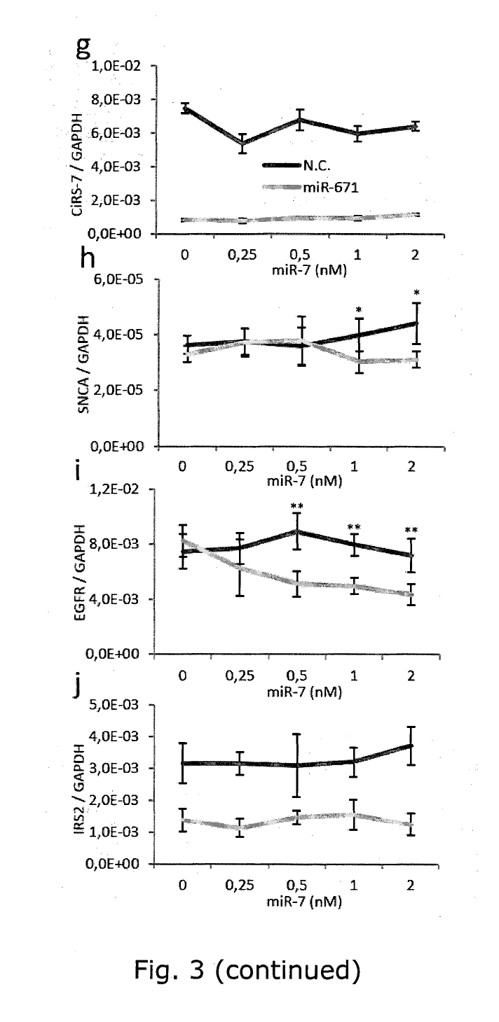

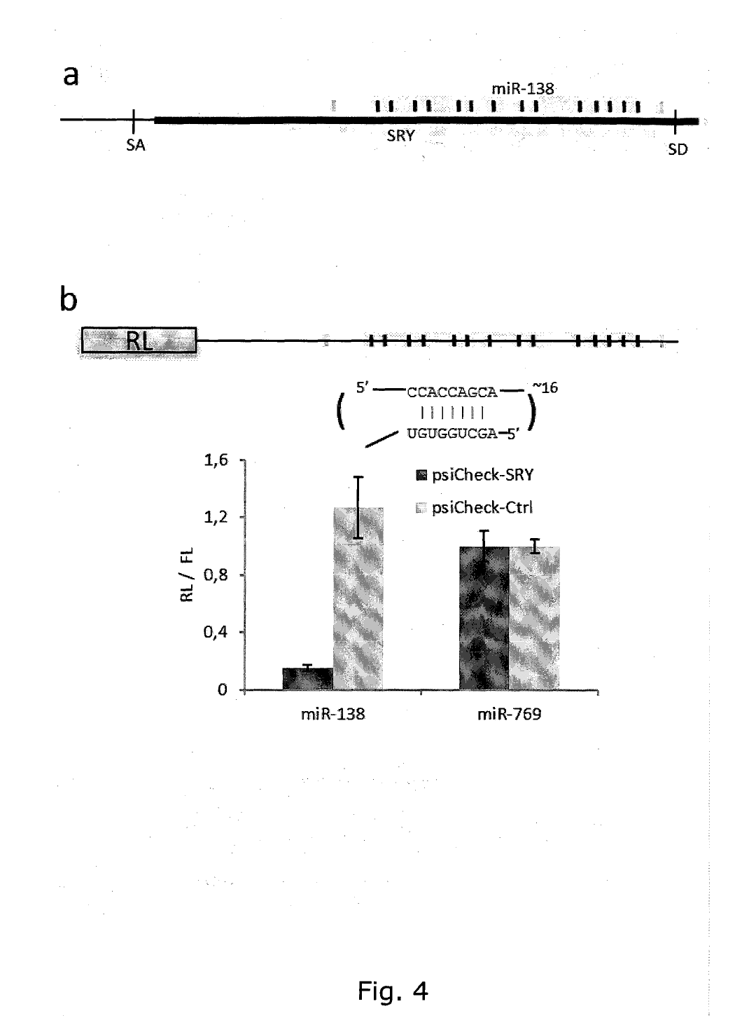

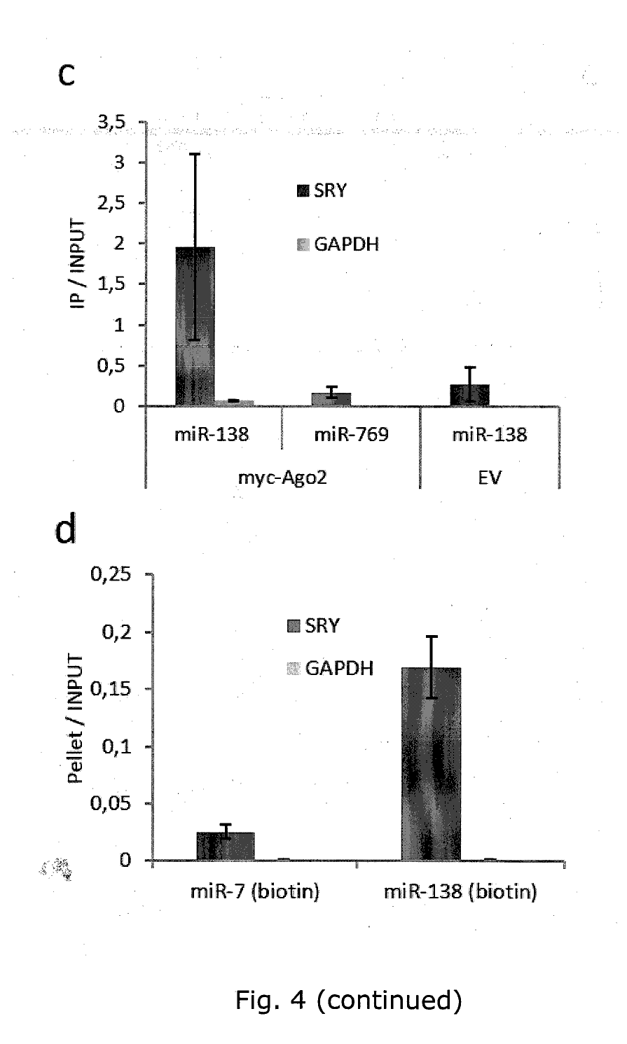

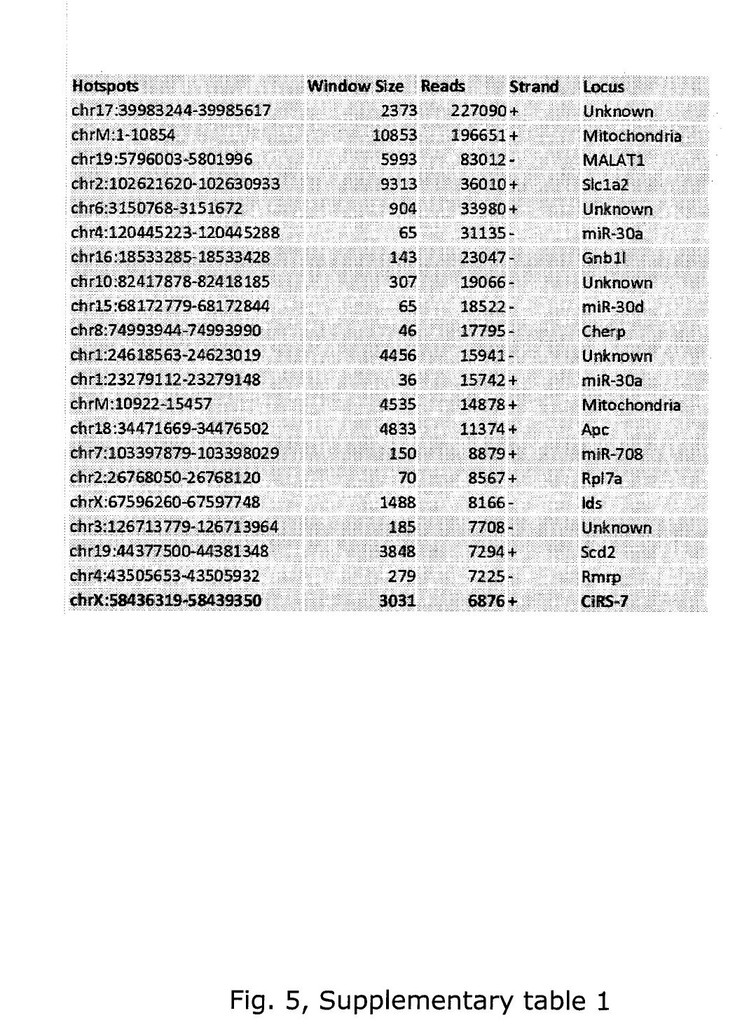

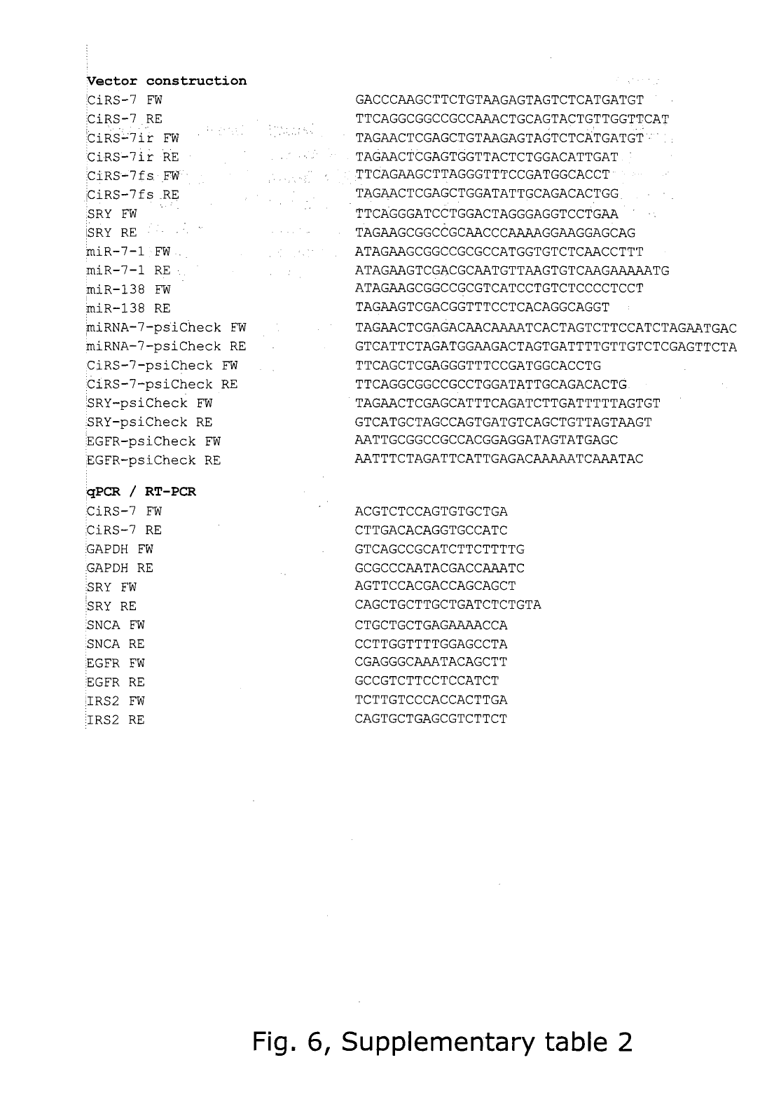

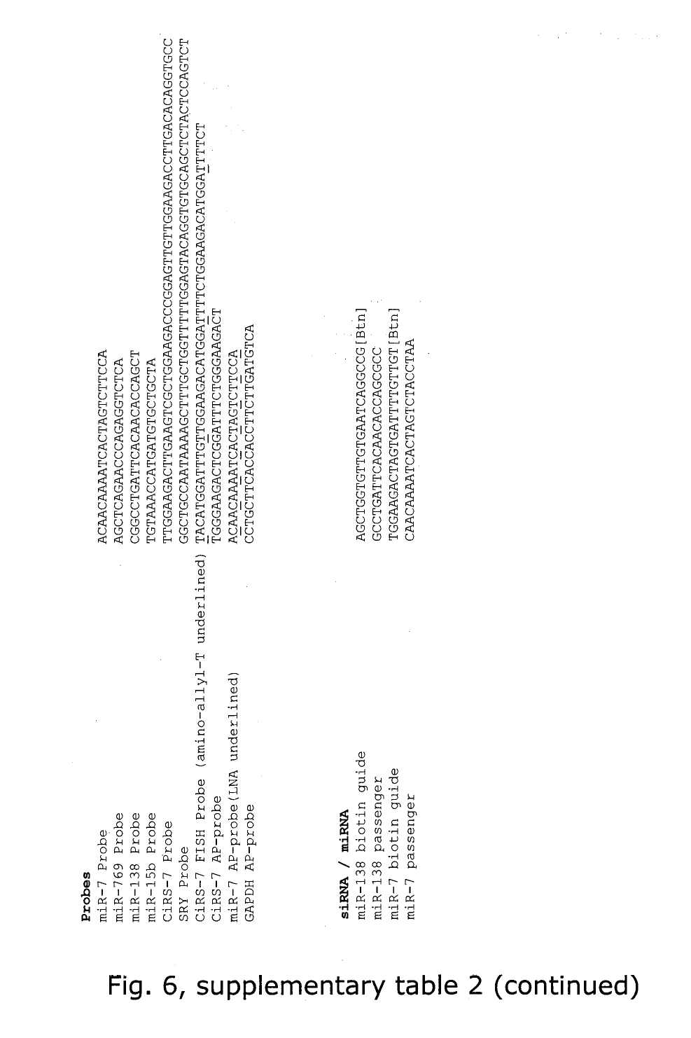

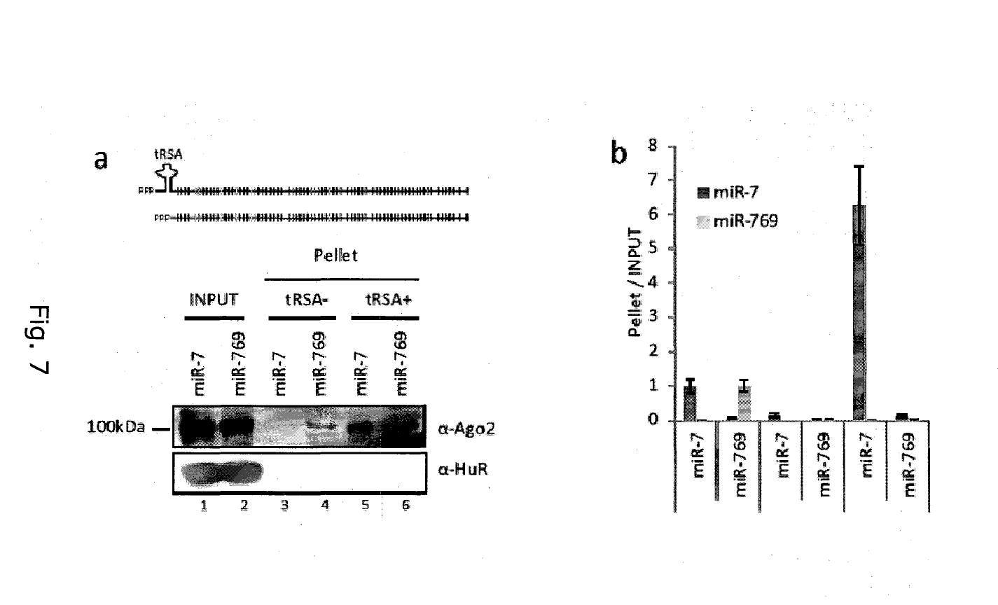

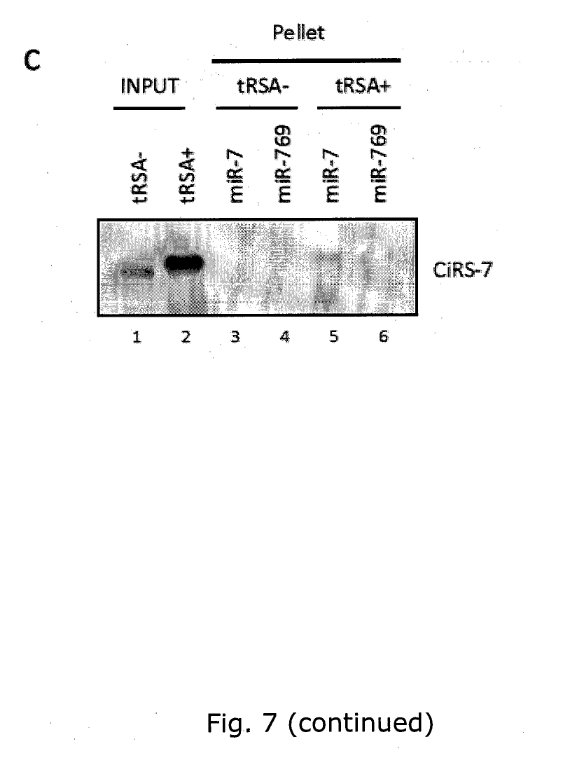

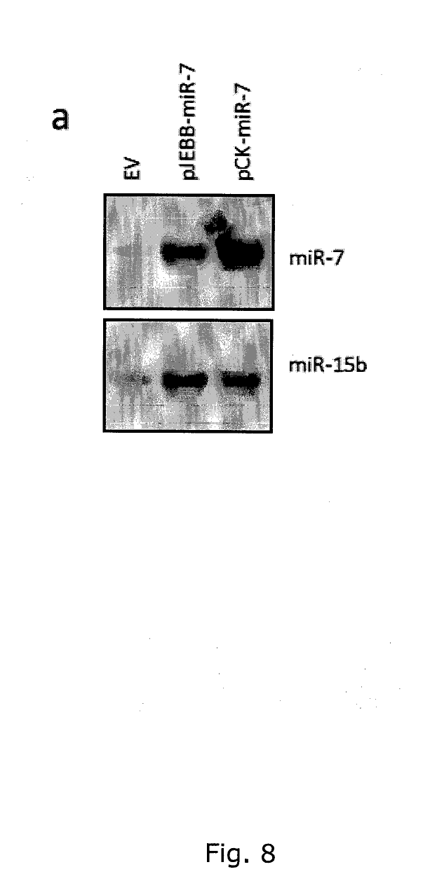

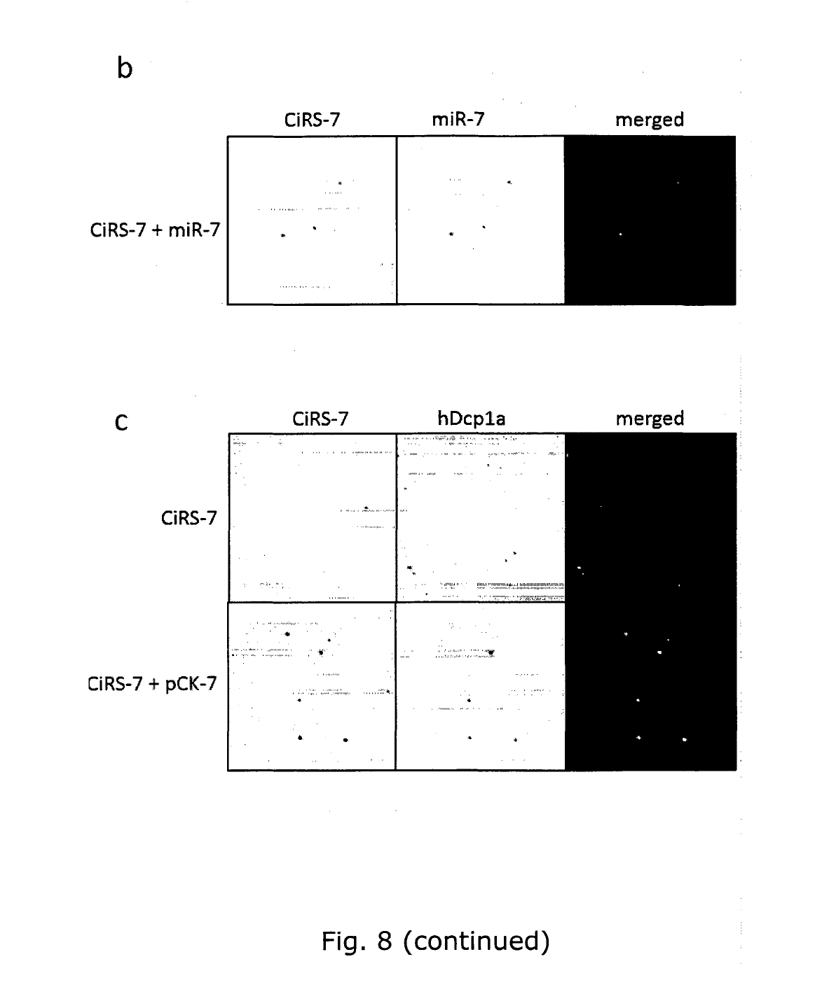

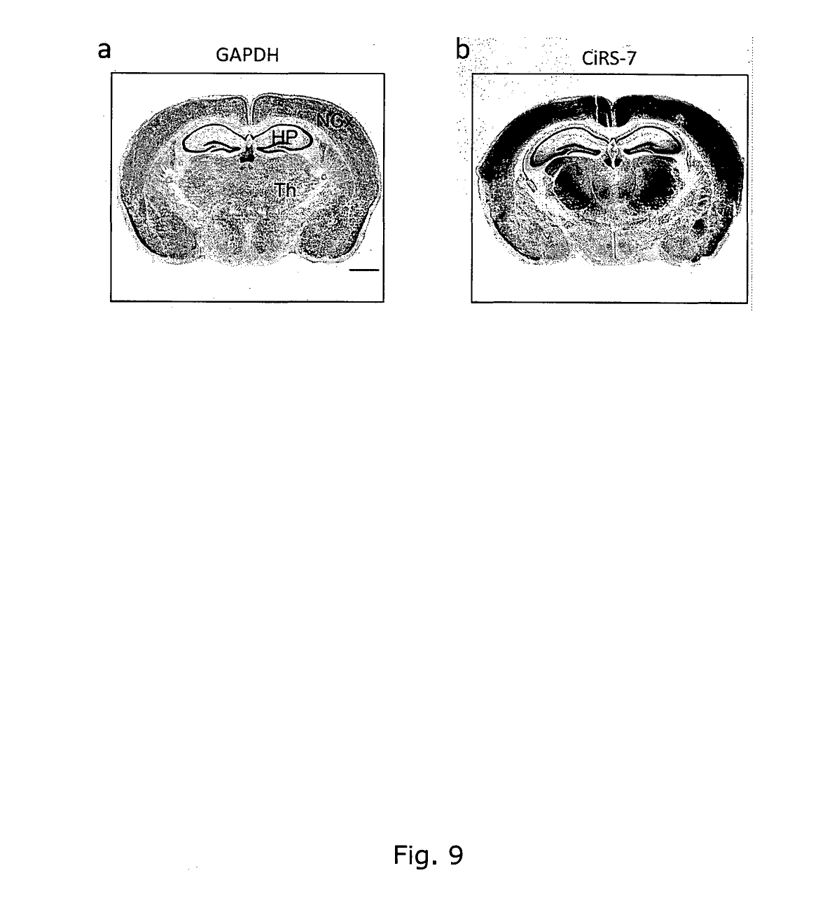

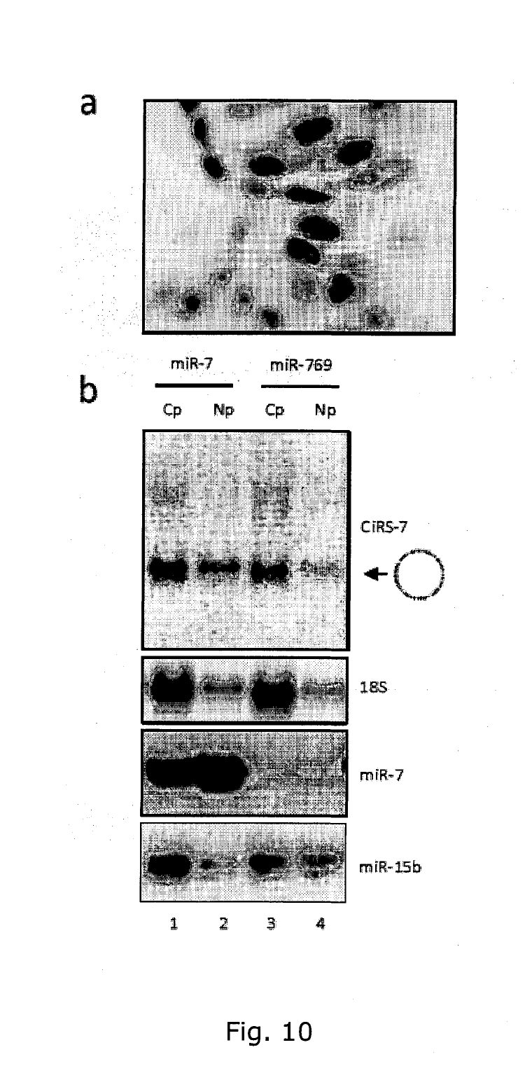

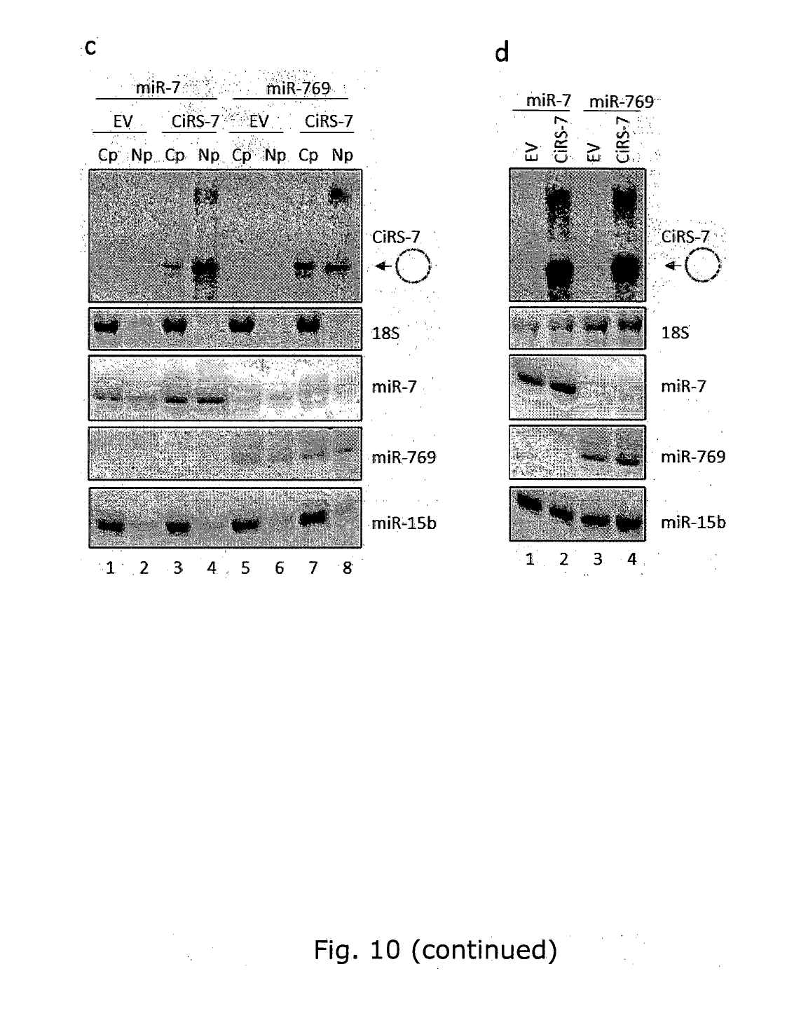

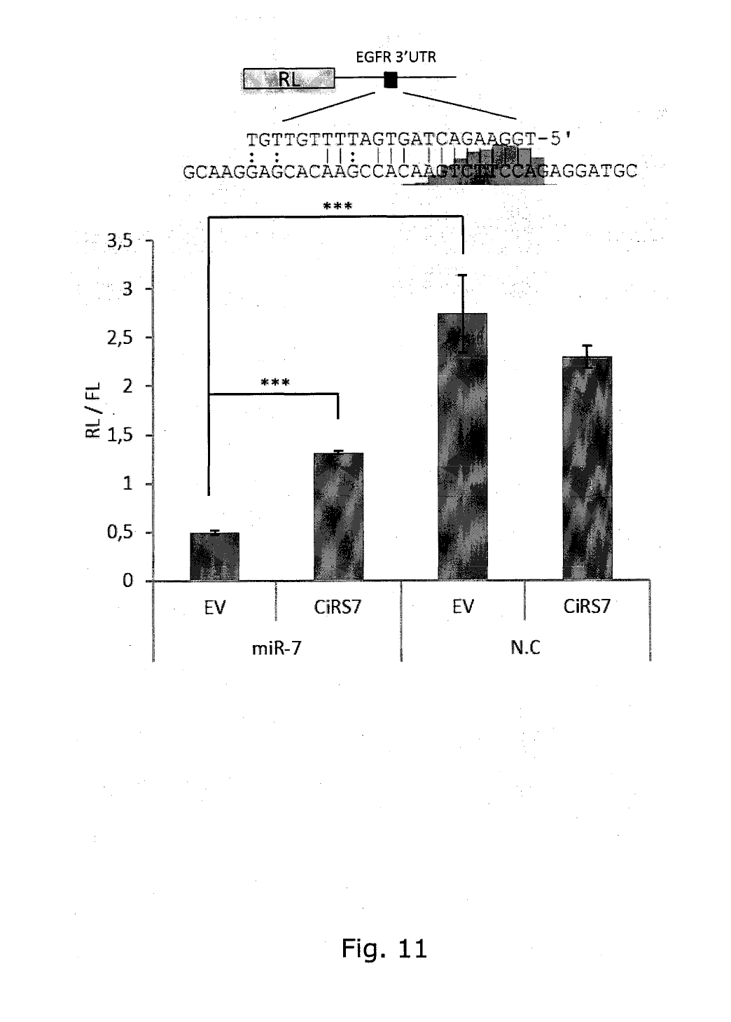

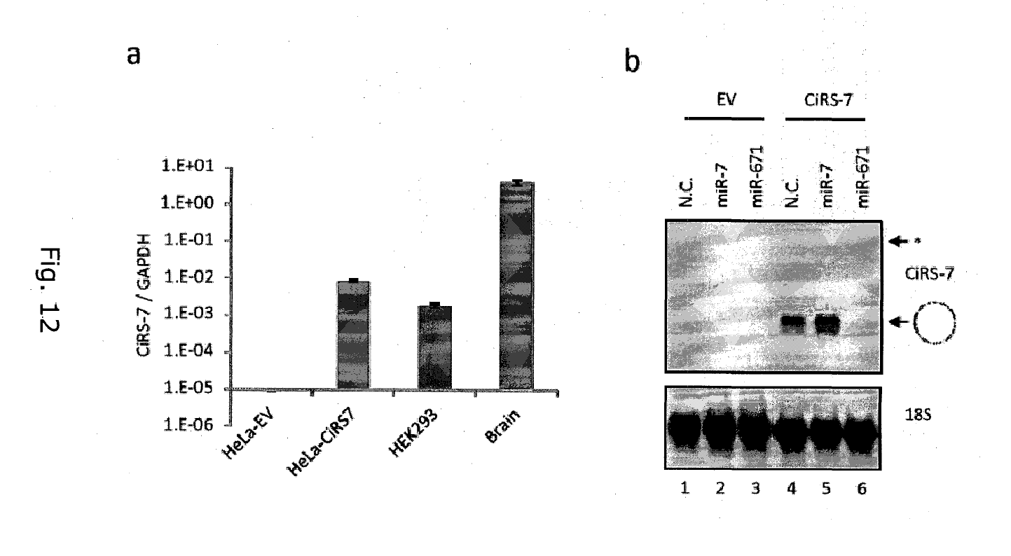

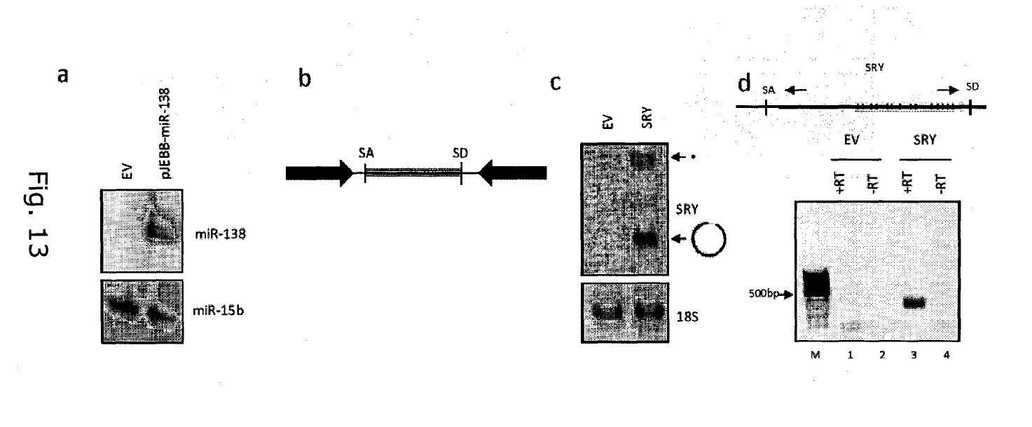

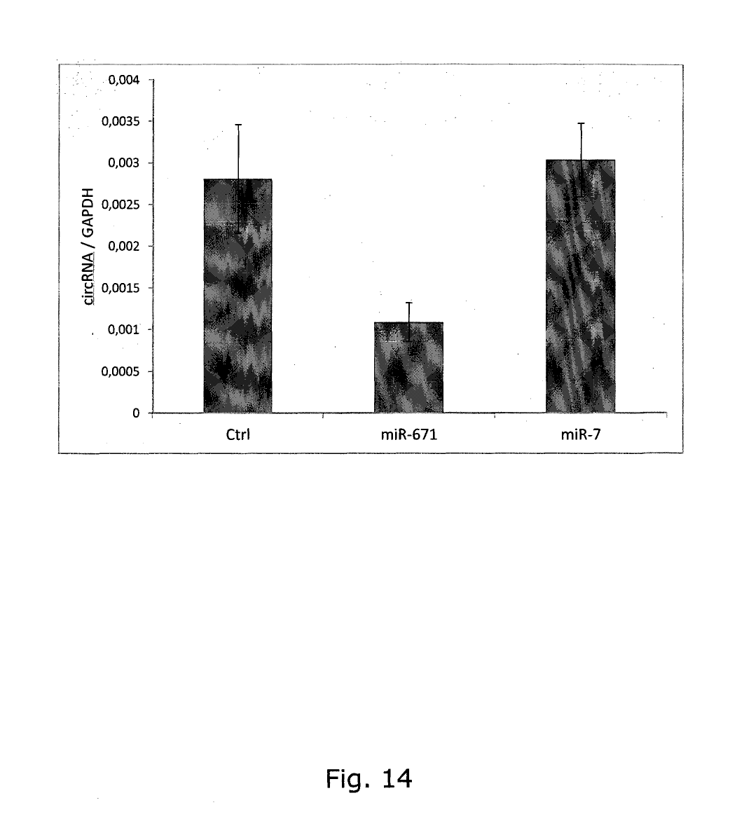

Circular SRY interacts with miR-138: a, Schematic representation of the SRY locus with splice sites (SA and SD) facilitating the SRY circularization and an open read frame depicted as the black bar. Perpendicular lines represent putative target sites for miR-138 (color code as in FIG. 1a). b, Luciferase reporter assays with the entire SRY sequence inserted into the 3′UTR of renilla luciferase. Forty eight hours after transfection with luciferase expression vectors (psiCheck-SRY or psiCheck-ctrl) and pJEBB-138 or pJEBB-769, luminescence was measured. The relative levels of renilla luminescence normalized to firefly are plotted. c, Immunoprecipitation (IP) of myc-tagged Ago2 from HEK293 cells co-transfected with SRY expression vector, myc-Ago2 or empty vector (EV) and pJEBB-138 or pJEBB-769 as denoted. SRY and GAPDH levels are quantified by RT-qPCR and the relative IP/Input ratios are plotted. d, As described in FIG. 2b, 3′ biotinylated miR-7 or miR-138 was transfected into HEK293 cells along with SRY expression vector. SRY and GAPDH levels were analysed in the input and bound fractions were evaluated by RT-qPCR.

FIG. 5, Supplementary Table S1

Ranked hotspot analysis of HITS-CLIP dataset. Hotspot analysis conducted on HITS-CLIP from mouse brain15. Each hotspot represents a cluster of reads with less than 50 bp distance to the closest neighbouring read.

FIG. 6, Supplementary Table S2: Primers and Probes.

FIG. 7, Supplementary Figure S1

Streptavidin-aptamer capture of Ago2 and miR-7. a, b, T7-transcripts covering the entire CiRS-7 sequence with or without a streptavidin aptamer (tRSA) sequence in the 5′ end (as schematically depicted above), were incubated with cell lysate pre-transfected with either pJEBB-7 or pJEBB-769. After streptavidin capture, the input and bound fraction were analysed by western blot using anti-myc antibody or anti-HuR (control) (a) or by miRNA Taqman® qPCR (normalized to INPUT) (b). c, Northern blot with RNA from T7 transcription prior and after biotin-capture probed with CiRS-7.

FIG. 8, Supplementary Figure S2

RNA-FISH and IF-FISH on HeLa cells. a, Northern blot with RNA from HEK293 cells transfected with empty vector (EV), pJEBB-7 or pCK-7 validating miRNA biogenesis from plasmid-based expression. The blot was probed against miR-7 and miR-15b (loading control). b, c, RNA-FISH and IF-FISH on HeLa cells transfected with constructs as indicated (as in FIG. 2c-d).

FIG. 9, Supplementary Figure S3

Sub-brain expression of CiRS-7 in mouse. a, b, Mouse brains sections hybridized with alkaline phosphatase-coupled GAPDH mRNA (a) or CiRS-7 (b) probe. HP, hippocampus; NCx, neocortex; Th, thalamus. Bar: 1 mm.

FIG. 10, Supplementary Figure S4

Subcellular localization of CiRS-7 and miR-7: a, Mouse brain section hybridized with alkaline phosphatase-coupled miR-7. b, Stable CiRS-7 expressing HEK293 cells transfected with miR-7 or miR-769 (pJEBB-7 or pJEBB-769, respectively). Forty eight hours after transfection, nuclear (Np) and cytoplasmic (Cp) RNA fractions were analysed by northern blotting. c, HEK293 cells were co-transfected with CiRS-7 or empty vector (EV) together with miR-7 or miR-769 and analysed as in (a). d, Total RNA from HEK293 cells were cotransfected with constructs as indicated and analysed by northern blotting.

FIG. 11, Supplementary Figure S5

Luciferase reporter assay. The 3′UTR of miR-7 target gene, EGFR, was inserted into psiCheck vector (schematic representation of target site depicted above). HEK293 cells were co-transfected with either 10 nM miR-7 or 10 nM N.C. (negative control) and pcDNA3-CiRS-7 or pcDNA3-EV. Forty eight hours after transfection, relative luminescence was measured and plotted.

FIG. 12, Supplementary Figure S6

CiRS-7 expression in stable HeLa cells. a, Northern blot using 10 ug RNA from HeLa-EV or Hela-CiRS-7 cells transfected with 10 nM N.C., miR-7 or miR-671. Blot was probed with CiRS-7 and 18S (loading control). The asterix (*) denotes a high-molecular, non-circular RNA species produced from the CiRS-7 construct. b, qRT-PCR on RNA from HeLa-EV, HeLa-CiRS-7, HEK293 cells, and total brain quantifying CiRS-7 levels relative to GAPDH mRNA.

FIG. 13, Supplementary Figure S7

miR-138 and SRY expression. a, Northern blot using 30 ug RNA from HEK293 cells transfected with either empty vector (EV, pJEBB) or pJEBB-138. Blot was probed against miR-138 (upper panel) and miR-15b (loading control, lower panel). b, Schematic representation of SRY expression vector (pcDNA3-SRY) in which the tet-responsive CMV promoter from the pcDNA5/FRT/TO vector has been inserted in an inverted orientation downstream the SRY locus. c, Northern blot using 10 ug RNA from HEK293 cells transfected with either empty vector (EV, pcDNA3) or the SRY expression construct (pcDNA3-SRY). Blot was probed with SRY and 18S (upper and lower panels, respectively). The asterix (*) denotes a high-molecular, non-circular RNA species produced from the SRY construct. d, RT-PCR on RNA from HEK293 cells transfected with either EV or SRY using primers depicted as arrows above.

FIG. 14 shows quantification of circular RNA (CiRS-7; with ˜70 sponge sites for miR-7 and a target site (cleavage site) for miR-671) levels in HEK293 cells after transfection with 10 nM non-related control microRNA (Ctrl), miR-7 or miR-671 mimics (Applied Biosystems) as indicated using Lipofectamine 2000 (Invitrogen). CiRS-7 levels are plotted relative to GAPDH.

The present invention will now be described in more detail in the following.

### DETAILED DESCRIPTION OF THE INVENTION

In the present disclosure the inventors present the idea of using circular RNA as efficient and stable miRNA inhibitors (also known as anti-miRs).

A microRNA (abbreviated miRNA) is a short ribonucleic acid (RNA) molecule found in eukaryotic cells.

A microRNA molecule has an average of 22 nucleotides.

miRNAs are post-transcriptional regulators that bind to partly complementary sequences on target messenger RNA transcripts (mRNAs), usually resulting in translational repression or target degradation and gene silencing.

miRNAs have also been reported to act as transcriptional silencers potentially through epigenetic effects.

The human genome encode over 1000 miRNAs, which target more than 70% of mammalian genes and are abundant in many human cell types.

The above mentioned approach has several advantages:

The circular nature of the RNA inhibitor will enable it to bind the miRNA but, remains resistant to miRNA destabilization mediated by for example but not limited to exonucleases. As a consequence the miRNA becomes unavailable for binding to and initiate degradation of their targeted RNAs such as mRNAs. Such target RNAs will thus be relieved of any repression caused by the said miRNA.

The sequences in the circular RNA can readily be exchanged to target any expressed miRNA of choice.

The circular RNA is stabilized against normal RNA degradation in the cells which usually occur from the termini of a transcript. Therefore, this approach does not depend on potentially toxic and artificial building blocks normally used to increase RNA stability in target cells.

Such RNAs with the capacity to inhibit interactions between a microRNA and its mRNA target are in the present context also called circular microRNA sponges and the effect is called “sponging”.

The inventors have devised a simple method, an insertion of inverted repeats flanking the sequence encoding the circular RNA, to produce large amounts circular RNA intracellularly from a conventional expression vector.

Circular RNAs are produced exploiting the natural machinery for RNA biogenesis.

They can be expressed from a DNA vector, other RNA molecules, transient or stable viral transduction, or from transgenes incorporated in the genome. Thus it is possible to use inducible or tissue-specific promoters for expression of circular RNA molecules.

It is possible to include multiple target sites against one or more miRNA within the circular RNA inhibitor and thereby tether and inactivate several different miRNAs at once using only one construct.

It is possible to control the presence of the circular RNA by targeting it with a miRNA or small interfering RNA perfectly complementary to the circular RNA. For example, a natural miRNA-671 exists that can cleave and quickly remove the circular RNA directed to microRNA-7.

This enables a tight control of circular RNA expression and an efficient antidote if a treatment based on circular RNAs is desired to be terminated.

### RNA Polynucleotide Characteristics

An aspect and an embodiment of the present invention relates to a polynucleotide RNA that comprises a sequence which is at least partly complementary to a human microRNA sequence, wherein said RNA polynucleotide is circular.

One embodiment of the present application relates to the polynucleotide of the present invention which comprises a region of contiguous nucleobase sequence which is 100% complementary to the human microRNA seed region.

In another embodiment of the present invention the polynucleotide may be single stranded or partly double stranded.

In animals miRNA complementarity typically encompasses the 5′ bases 2-7 of the microRNA, the microRNA seed region, and one miRNA can therefore target many different sites on the same mRNA or on many different mRNAs.

MicroRNAs are therefore promiscuous.

The sequence which is at least partly complementary or 100% complementary to a human microRNA sequence is also called the anti-miR sequence or blockmir sequence.

Another embodiment of the present invention relates to the RNA polynucleotide of the present invention where at least part of the polynucleotide RNA is complementary to the mature human microRNA sequence.

Another embodiment of the present invention relates to a polynucleotide comprising an antisense sequence to the seed sequence of a microRNA, wherein the polynucleotide is circular.

The polynucleotide of the present invention can be purified or isolated from a sample, a cell culture or similar.

Thus, in a further embodiment of the present invention is the RNA polynucleotide of the present invention an isolated and/or purified polynucleotide.

In yet another embodiment of the present invention the RNA polynucleotide is capable of recruiting the RNAi machinery.

In another embodiment of the present invention the RNA polynucleotide is not capable of recruiting the RNAi machinery.

Herein the RNAi machinery refers to the cellular components necessary for the activity of siRNAs and microRNAs or for the RNAi pathway.

A major player of the RNAi machinery is the RNA induced silencing complex (the RISC complex).

Herein, nucleobase, base, nucleic acid, refer to the base moiety of a nucleotide unit. The nucleotide unit may be of DNA, RNA, INA, LNA, 2′F and 2′MeO modified RNA or any other nucleic acid or any nucleic acid capable of specific base pairing. The nucleotide unit may also be part of PNA (peptide nucleic acid).

In some embodiments, the nucleotide unit may be a universal nucleotide unit.

When referring the length of a sequence, reference may be made to the number of nucleotide units or to the number of bases.

When referring to a complementary sequence, G pairs to C, A pairs to T and U and vice versa. In a preferred embodiment, G also pairs to U and vice versa to form a so-called wobble base pair.

A wobble base pair is a non-Watson-Crick base pairing between two nucleotides in RNA molecules.

The four main wobble base pairs are guanine-uracil, inosine-uracil, inosine-adenine, and inosine-cytosine (G-U, I-U, I-A and I-C).

Thus, in another preferred embodiment, the base inosine (I) may comprise a part of a microRNA or any other polynucleotide of the invention and might optionally base pair to A, C and U. In still another preferred embodiment, universal bases may be used.

Universal bases can typically base pair to G, C, A, U and T.

Often universal bases do not form hydrogen bonds with the opposing base on the other strand.

In still another preferred embodiment, a complementary sequence refers to a contiguous sequence exclusively of Watson-Crick base pairs.

The polynucleotide of the invention may also recruit RNase H to mediate cleavage of the target RNA.

A lariat circle is formed during RNA spicing by the 2′OH of a specific adenosine in the intron attacking the 5′ splice site, thereby forming the said lariat structure.

The invention also provides methods for providing microRNA targets of target RNAs, methods for validating microRNA target regions of target RNAs and methods of modulating the activity of target RNAs using polynucleotides of the invention.

One aspect and an embodiment of the present invention provides a circular polynucleotide RNA comprising an antisense sequence that comprises a guide sequence corresponding partially to the sequence of a microRNA, with the proviso that the polynucleotide is not a microRNA.

Such a polynucleotide is of interest because it can be used to block the target region of a target RNA, said target region being involved in microRNA regulation of the target RNA, thereby relieving the target RNA from the repression of specific miRNAs.

The circular polynucleotide of the invention can also inhibit or block any other RNA in the cells. Therefore, in another preferred embodiment, the circular polynucleotide of the invention can be used to inhibit ribosomal RNAs, tRNAs, snRNAs, snoRNAs, siRNA, long non-coding RNA, piRNA and specific mRNA, referred to as RNA blocker. In this preferred embodiment, the circular polynucleotide of the invention can also block miRNA target sites relieving the target from miRNA-mediated repression.

The inhibitory effect of the circular polynucleotide of the invention is not limited to RNA. Providing one or more binding sites for proteins can lead to a sequestering and inactivation of protein function referred to as protein decoy. Another preferred embodiment is to insert RNA sequences that bind cellular proteins with the aim to inactivate them. Yet another aspect and an embodiment is to use the circular polynucleotide of the invention as a scaffold to sequester proteins to enhance function, e.g. by dimerization or multimerization of the same protein or by bringing multiple proteins together to enhance function.

In a preferred embodiment, the circular polynucleotide of the invention comprise a sequence selected from the group consisting of sequences that are capable of base pairing to the complementary sequence of a sequence selected from the group consisting of position 1-22, position 1-21, position 1-20, position 1-19, position 1-18, position 1-17, position 1-16, position 1-15, position 1-14, position 1-13, position 1-12, position 1-11, position 1-10, position 1-9, position 1-8, position 1-7, position 1-6, position 2-22, position 2-21, position 2-20, position 2-19, position 2-18, position 2-17, position 2-16, position 2-15, position 2-14, position 2-13, position 2-12, position 2-11, position 2-10, position 2-9, position 2-8, position 2-7, position 3-22, position 3-21, position 3-20, position 3-19, position 3-18, position 3-17, position 3-16, position 3-15, position 3-14, position 3-13, position 3-12, position 3-11, position 3-10, position 3-9, position 3-8, position 4-22, position 4-21, position 4-20, position 4-19, position 4-18, position 4-17, position 4-16, position 4-15, position 4-14, position 4-13, position 4-12, position 4-11, position 4-10, position 4-9, position 5-22, position 5-21, position 5-20, position 5-19, position 5-18, position 5-17, position 5-16, position 5-15, position 5-14, position 5-13, position 5-12, position 5-11, position 5-10, position 6-22, position 6-21, position 6-20, position 6-19, position 6-18, position 6-17, position 6-16, position 6-15, position 6-14, position 6-13, position 6-12, position 6-11, position 7-22, position 7-21, position 7-20, position 7-19, position 7-18, position 7-17, position 7-16, position 7-15, position 7-14, position 7-13, position 7-12, position 8-22, position 8-21, position 8-20, position 8-19, position 8-18, position 8-17, position 8-16, position 8-15, position 8-14, position 8-13, position 9-22, position 9-21, position 9-20, position 9-19, position 9-18, position 9-17, position 9-16, position 9-15, position 9-14, position 10-22, position 10-21, position 10-20, position 10-19, position 10-18, position 10-17, position 10-16, position 10-15, position 11-22, position 11-21, position 11-20, position 11-19, position 11-18, position 11-17, position 11-16, position 12-22, position 12-21, position 12-20, position 12-19, position 12-18, position 12-17, position 13-22, position 13-21, position 13-20, position 13-19, position 13-18, position 14-22, position 14-21, position 14-20, position 14-19, position 15-22, position 15-21, position 15-20, position 16-22, position 16-21, position 17-22″ of a human microRNA, preferably defined by any of SEQ ID NOs: 51-86.

In a preferred embodiment, the circular polynucleotide of the invention comprise a sequence selected from the group consisting of sequences that are capable of base pairing to the complementary sequence of a sequence selected from the group consisting of position 1-22, position 1-21, position 1-20, position 1-19, position 1-18, position 1-17, position 1-16, position 1-15, position 1-14, position 1-13, position 1-12, position 1-11, position 1-10, position 1-9, position 1-8, position 1-7, position 1-6, position 2-22, position 2-21, position 2-20, position 2-19, position 2-18, position 2-17, position 2-16, position 2-15, position 2-14, position 2-13, position 2-12, position 2-11, position 2-10, position 2-9, position 2-8, position 2-7, position 3-22, position 3-21, position 3-20, position 3-19, position 3-18, position 3-17, position 3-16, position 3-15, position 3-14, position 3-13, position 3-12, position 3-11, position 3-10, position 3-9, position 3-8, position 4-22, position 4-21, position 4-20, position 4-19, position 4-18, position 4-17, position 4-16, position 4-15, position 4-14, position 4-13, position 4-12, position 4-11, position 4-10, position 4-9, position 5-22, position 5-21, position 5-20, position 5-19, position 5-18, position 5-17, position 5-16, position 5-15, position 5-14, position 5-13, position 5-12, position 5-11, position 5-10, position 6-22, position 6-21, position 6-20, position 6-19, position 6-18, position 6-17, position 6-16, position 6-15, position 6-14, position 6-13, position 6-12, position 6-11, position 7-22, position 7-21, position 7-20, position 7-19, position 7-18, position 7-17, position 7-16, position 7-15, position 7-14, position 7-13, position 7-12, position 8-22, position 8-21, position 8-20, position 8-19, position 8-18, position 8-17, position 8-16, position 8-15, position 8-14, position 8-13, position 9-22, position 9-21, position 9-20, position 9-19, position 9-18, position 9-17, position 9-16, position 9-15, position 9-14, position 10-22, position 10-21, position 10-20, position 10-19, position 10-18, position 10-17, position 10-16, position 10-15, position 11-22, position 11-21, position 11-20, position 11-19, position 11-18, position 11-17, position 11-16, position 12-22, position 12-21, position 12-20, position 12-19, position 12-18, position 12-17, position 13-22, position 13-21, position 13-20, position 13-19, position 13-18, position 14-22, position 14-21, position 14-20, position 14-19, position 15-22, position 15-21, position 15-20, position 16-22, position 16-21, position 17-22″ of a human microRNA, preferably defined by any of SEQ ID NOs: 51-86 and are not capable of forming a consecutive base pair with the neighbouring nucleotide of either side of the aforementioned positions.

The phrase “are capable of base pairing to” is related to the term complementary sequence. I.e. a first sequence is capable of base pairing to a second sequence, which is complementary to the first sequence.

In another preferred embodiment, the circular polynucleotide of the invention consists of an antisense sequence selected from the group consisting of sequences that are capable of base pairing to the sequence or the complementary sequence of a sequence selected from the group consisting selected from the group consisting of position 1-22, position 1-21, position 1-20, position 1-19, position 1-18, position 1-17, position 1-16, position 1-15, position 1-14, position 1-13, position 1-12, position 1-11, position 1-10, position 1-9, position 1-8, position 1-7, position 1-6, position 2-22, position 2-21, position 2-20, position 2-19, position 2-18, position 2-17, position 2-16, position 2-15, position 2-14, position 2-13, position 2-12, position 2-11, position 2-10, position 2-9, position 2-8, position 2-7, position 3-22, position 3-21, position 3-20, position 3-19, position 3-18, position 3-17, position 3-16, position 3-15, position 3-14, position 3-13, position 3-12, position 3-11, position 3-10, position 3-9, position 3-8, position 4-22, position 4-21, position 4-20, position 4-19, position 4-18, position 4-17, position 4-16, position 4-15, position 4-14, position 4-13, position 4-12, position 4-11, position 4-10, position 4-9, position 5-22, position 5-21, position 5-20, position 5-19, position 5-18, position 5-17, position 5-16, position 5-15, position 5-14, position 5-13, position 5-12, position 5-11, position 5-10, position 6-22, position 6-21, position 6-20, position 6-19, position 6-18, position 6-17, position 6-16, position 6-15, position 6-14, position 6-13, position 6-12, position 6-11, position 7-22, position 7-21, position 7-20, position 7-19, position 7-18, position 7-17, position 7-16, position 7-15, position 7-14, position 7-13, position 7-12, position 8-22, position 8-21, position 8-20, position 8-19, position 8-18, position 8-17, position 8-16, position 8-15, position 8-14, position 8-13, position 9-22, position 9-21, position 9-20, position 9-19, position 9-18, position 9-17, position 9-16, position 9-15, position 9-14, position 10-22, position 10-21, position 10-20, position 10-19, position 10-18, position 10-17, position 10-16, position 10-15, position 11-22, position 11-21, position 11-20, position 11-19, position 11-18, position 11-17, position 11-16, position 12-22, position 12-21, position 12-20, position 12-19, position 12-18, position 12-17, position 13-22, position 13-21, position 13-20, position 13-19, position 13-18, position 14-22, position 14-21, position 14-20, position 14-19, position 15-22, position 15-21, position 15-20, position 16-22, position 16-21, position 17-22″ of a human microRNA, preferably defined by any of SEQ ID NOs: 51-86.

Preferably, the polynucleotide of the invention comprises an antisense or sense sequence that comprises a contiguous stretch of nucleotides, complementary or identical to the micro RNA target sequence of a target RNA selected from the group consisting of: less than 60 bases, less than 50 bases, less than 40 bases, less than 39 bases, less than 38 bases, less than 37 bases, less than 36 bases, less than 35, less than 34 bases, less than 33 bases, less than 32 bases, less than 31 bases, bases, less than 30 bases, less than 29 bases, less than 28 bases, less than 27 bases, less than 26 bases, less than 25 bases, less than 24 bases, less than 23 bases, less than 22 bases, less than 21 bases, less than 20 bases, less than 19 bases, less than 18 bases, less than 17 bases, less than 16 bases, less than 15 bases, less than 14 bases, less than 13 bases, less than 12 bases, less than 11 bases, less than 10 bases, less than 9 bases, less than 8 bases, less than 7 bases, more than 60 bases, more than 50 bases, more than 40 bases, more than 39 bases, more than 38 bases, more than 37 more, more than 36 bases, more than 35, more than 34 bases, more than 33 bases, more than 32 bases, more than 31, more than 30 bases, more than 29 bases, more than 28 bases, more than 27 bases, more than 26 bases, more than 25 bases, more than 24 bases, more than 23 bases, more than 22 bases, more than 21 bases, more than 20 bases, more than 19 bases, more than 18 bases, more than 17 bases, more than 16 bases, more than 15 bases, more than 14 bases, more than 13 bases, more than 12 bases, more than 11 bases, more than 10 bases, more than 9 bases, more than 8 bases, more than 7 bases, more than 6 bases and more than 5 bases.

A contiguous stretch of bases is intended to mean a non-interrupted sequence of bases that all fit into a duplex formed between the polynucleotide of the invention and the target RNA. I.e. there are preferably no bulges in the duplex and it is preferred that the sequences are complementary (see the definition of complementary sequences above).

Most preferred is perfect Watson-Crick duplex between the polynucleotide of the invention and target region of the target RNA.

The terms contiguous and continuous are used interchangeably herein.

In another embodiment, the polynucleotides of the invention comprise an antisense or sense sequence that comprises a contiguous stretch of bases complementary to the microRNA target sequence of a target RNA, said contiguous stretch of bases being selected from the group consisting of between 10 and 14 bases, between 12 and 16 bases, between 14 and 18 bases, between 16 and 20, between 10 and 25 bases, between 12 and 24 bases, between 14 and 22 bases, between 15 and 22 bases and between 15 and 20 bases.

More preferred is a contiguous stretch of bases between 7 and 25 bases.

Most preferred is several contiguous stretches of bases between 5 and 20 bases.

Also preferred is an adenosine (A) residue in the polynucleotide opposing the 5′ nucleotide in the microRNA.

The preferred microRNA targeted by the present polynucleotides are: miR-7-5p (SEQ ID NO 51), miR-7-1-3p (SEQ ID NO 52), miR-7-2-3p (SEQ ID NO 53), miR-21-5p (SEQ ID NO 54), miR-21-3p (SEQ ID NO 55), miR-96-5p (SEQ ID NO 56), miR-96-3p (SEQ ID NO 57), miR-185-5p (SEQ ID NO 58), miR-185-3p (SEQ ID NO 59), miR-324-5p (SEQ ID NO 60), miR-324a-3p (SEQ ID NO 61), miR-133b (SEQ ID NO 62), miR-205-5p (SEQ ID NO 63), miR-205-3p (SEQ ID NO 64), miR-200a-5p (SEQ ID NO 65), miR-200a-3p (SEQ ID NO 66), miR-200b-5p (SEQ ID NO 67), miR-200b-3p (SEQ ID NO 68), miR-200c-5p (SEQ ID NO 69), miR-200c-3p (SEQ ID NO 70), miR-141-5p (SEQ ID NO 71), miR-141-3p (SEQ ID NO 72), miR-429 (SEQ ID NO 73), miR-132-5p (SEQ ID NO 74), miR-132-3p (SEQ ID NO 75), miR-134 (SEQ ID NO 76), miR-124-5p (SEQ ID NO 77), miR-124-3p (SEQ ID NO 78), miR-155-5p (SEQ ID NO 79), miR-155-3p (SEQ ID NO 80), miR-221-5p (SEQ ID NO 81), miR-221-3p (SEQ ID NO 82), miR-222-5p (SEQ ID NO 83), miR-222-3p (SEQ ID NO 84), miR-122-5p (SEQ ID NO 85), miR-122-3p (SEQ ID NO 86).

### Sequence Identity

The polynucleotide of the present invention can also be defined by the sequence identity with a human microRNA or the complementary sequence of a human microRNA.

Two sequences that are complementary at less than 100% of the sequence are defined as being at least partly complementary.

As commonly defined “identity” is here defined as sequence identity between genes or proteins at the nucleotide, base or amino acid level, respectively. Specifically a DNA and an RNA sequence are considered identical if the transcript of the DNA sequence can be transcribed to the identical RNA sequence.

Thus, in the present context “sequence identity” is a measure of identity between proteins at the amino acid level and a measure of identity between nucleic acids at nucleotide level.

The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned.

To determine the percent identity of two amino acid sequences or of two nucleic acids, the sequences are aligned for optimal comparison purposes (e.g., gaps may be introduced in the sequence of a first amino acid or nucleic acid sequence for optimal alignment with a second amino or nucleic acid sequence).

The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences (i.e., % identity=# of identical positions/total # of positions (e.g., overlapping positions)×100).

In one embodiment the two sequences are the same length.

In another embodiment the two sequences are of different length and gaps are seen as different positions.

One may manually align the sequences and count the number of identical amino acids. Alternatively, alignment of two sequences for the determination of percent identity may be accomplished using a mathematical algorithm. Such an algorithm is incorporated into the NBLAST and XBLAST programs of (Altschul et al. 1990). BLAST nucleotide searches may be performed with the NBLAST program, score=100, wordlength=12, to obtain nucleotide sequences homologous to a nucleic acid molecules of the invention. BLAST protein searches may be performed with the XBLAST program, score=50, wordlength=3 to obtain amino acid sequences homologous to a protein molecule of the invention.

To obtain gapped alignments for comparison purposes, Gapped BLAST may be utilised.

Alternatively, PSI-Blast may be used to perform an iterated search which detects distant relationships between molecules. When utilising the NBLAST, XBLAST, and Gapped BLAST programs, the default parameters of the respective programs may be used. See http://www.ncbi.nlm.nih.gov. Alternatively, sequence identity may be calculated after the sequences have been aligned e.g. by the BLAST program in the EMBL database (www.ncbi.nlm.gov/cgi-bin/BLAST). Generally, the default settings with respect to e.g. “scoring matrix” and “gap penalty” may be used for alignment. In the context of the present invention, the BLASTN and PSI BLAST default settings may be advantageous.

The percent identity between two sequences may be determined using techniques similar to those described above, with or without allowing gaps. In calculating percent identity, only exact matches are counted.

An embodiment of the present invention thus relates to sequences of the present invention that has some degree of sequence variation.

In one embodiment of the present invention has the sequence identity of the anti-miR or blockmir sequence comprised within the polynucleotide of the present invention 100% sequence identity with the anti-miR or the microRNA sequence, such as at least 99%, such as at least 95%, such as at least 90%, such as at least 85%, such as at least 80%, such as at least 75%, such as at least 70%, such as at least 65%, such as at least 60%, such as at least 55%, such as at least 50%, such as at least 45%, such as at least 40%, such as at least 35%, such as at least 30%, such as at least 25% sequence identity.

In another embodiment of the present invention are 7 out of 7 nucleotides at the 5′ end overlapping, such as at least 6 out of 7, such as at least 5 out of 7, such as at least 4 out of 7, such as at least 3 out of 7, such as at least 2 out of 7 nucleotides at the 5′ end.

In one embodiment of the present invention refers this complementarity to the match between the seed or anti-seed sequence of the microRNA and the sequence comprised within the polynucleotide which is at least party complementary.

It should be noted that while the sequences in the present application are both RNA and DNA sequences, the present invention contemplates the corresponding RNA sequence, and DNA and RNA complementary sequences as well.

Thus, in cases where a DNA sequence is mentioned refers such DNA sequence also to the RNA equivalent i.e. with Ts exchanged with Us as well as their complimentary sequences.

### Cassettes, Vectors and Expression of RNA of the Invention

An expression vector, otherwise known as an expression construct, is generally a plasmid or a virus or virus derived vector (such as but not limited to an adenoviral, retroviral, baculoviral or lentiviral vectors) that is used to introduce a specific gene (in the present context the DNA molecule of the present invention) into a target cell.

Once the expression vector is inside the cell, it can express an RNA which subsequently may be translated into a protein. The circular RNA product will usually not be translated due to the lack of 5′-cap and poly-A tail. However, introduction of internal ribosome binding sites will potentially enable protein production from circular RNA products.

However, in the present case the expression vector may encode the RNA of the present invention which does not encode a protein because there is no poly-A tail.

Thus, one aspect and an embodiment of the present invention relates to an expression vector which encodes a RNA that cannot be translated into a protein.

The plasmid is frequently engineered to contain regulatory sequences that act as enhancer and promoter regions and lead to efficient transcription of the gene carried on the expression vector.

The goal of a well-designed expression vector is the production of sufficient amounts of stable circular RNA to exert an effect as microRNA inhibitor, target RNA blocker or as protein decoy.

Expression vectors are basic tools for biotechnology and the production of proteins such as insulin that are important for medical treatments of specific diseases like diabetes.

Thus, an aspect and an embodiment of the present invention therefore relates to an expression vector comprising a DNA molecule as described above.

A cassette is the expression vector that is prepared for insertion or the DNA molecule of the present invention.

Thus, one embodiment of the present invention relates to a cassette that is capable of expressing the RNA molecule of the present invention.

The cassette or expression vector of the present invention may be several sizes. Thus, in one embodiment of the present invention is the size of the cassette or expression vector less than 5 kb, such as less than 3 kb, such as less than 2 kb, such as less than 1 kb.

In another embodiment of the present invention is the size 200-1500 bases, such as 300-1200 bases, such as 400-1000 bases, such as 500-750 bases.

Another embodiment the present invention provides a cassette vector for expressing the nucleic acid sequences of the present invention, comprising, inserted therein, the nucleic acid sequence according to the invention and having an active promoter upstream thereof.

An aspect and an embodiment of the present invention relates to a DNA polynucleotide comprising a promoter, a nucleic acid sequence that is complementary to a human microRNA sequence, a splice acceptor site (SA) upstream of the nucleic acid sequence that is complementary to a human microRNA sequence, and a splice donor site (SD) downstream of the nucleic acid sequence that is complementary to a human microRNA sequence.

Another aspect and an embodiment of the present invention relates to a DNA polynucleotide comprising a promoter a nucleic acid sequence that is a guide sequence corresponding to the seed sequence of a human microRNA sequence, a splice acceptor site (SA) upstream of the nucleic acid sequence that is a guide sequence corresponding to the seed sequence of a human microRNA sequence, and a splice donor site (SD) downstream of the nucleic acid sequence that is a guide sequence corresponding to the seed sequence of a human microRNA sequence.

The DNA of the present invention may comprise two or more of the nucleic acids of the present invention.

Thus, a further aspect and an embodiment of the present invention relates to a DNA polynucleotide comprising a promoter, two or more nucleic acid sequences that is selected from the group consisting of a nucleic acid sequence that is complementary to a human microRNA sequence and a nucleic acid sequence that is a guide sequence corresponding to the seed sequence of a human microRNA sequence, a splice acceptor site (SA) upstream of the two or more nucleic acid sequences that is selected from the group consisting of a nucleic acid sequence that is complementary to a human microRNA sequence and a nucleic acid sequence that is a guide sequence corresponding to the seed sequence of a human microRNA sequence, and a splice donor site (SD) downstream of the two or more nucleic acid sequences that is selected from the group consisting of a nucleic acid sequence that is complementary to a human microRNA sequence and a nucleic acid sequence that is a guide sequence corresponding to the seed sequence of a human microRNA sequence.

Yet another aspect and an embodiment of the present invention relates to a DNA polynucleotide comprising a promoter, a nucleic acid sequence that acts as inhibitor of RNA or protein, a splice acceptor site (SA) upstream of the nucleic acid sequence that is a sequence acting as an RNA blocker or protein decoy, and a splice donor site (SD) downstream of the nucleic acid sequence that is a sequence acting as inhibitor of RNA or protein.

Yet another aspect and an embodiment of the present invention relates to a DNA polynucleotide comprising a promoter, a nucleic acid sequence that acts as inhibitor of RNA or protein, a splice donor (SD) upstream of the nucleic acid sequence that is a sequence acting as an RNA blocker or protein decoy, and a splice acceptor site (SA) downstream of the nucleic acid sequence that is a sequence acting as inhibitor of RNA or protein.

The DNAs, vector or cassettes of the present invention may comprise one or more introns.

In one embodiment of the present invention the DNAs, vector or cassettes comprises at least one intron.

An inverted repeat (or IR) is a sequence of nucleotides that is the reversed complement of another sequence further downstream, for example, 5′-GACTGC . . . GCAGTC-3′.

When no nucleotides intervene between the sequence and its downstream complement, it is called a palindrome.

Inverted repeats define the boundaries in transposons and retroviruses.

Inverted repeats also indicate regions capable of self-complementary base pairing (regions within a single sequence which can base pair with each other).

Thus, in one embodiment of the present invention the DNA polynucleotide of the present invention further comprises inverted repeats.

Another embodiment of the present invention relates to an RNA which transcribed from the DNA of the present invention.

Another embodiment of the present invention relates to inverted repeats positioned in the DNA in part or fully outside the RNA coding segment.

A further embodiment of the present invention relates to an RNA of the present invention which is capable of splicing.

In another embodiment of the present invention the DNA polynucleotide does not comprise a microRNA or does not comprise a sequence corresponding to the complete sequence of a microRNA.

The RNA or DNA polynucleotides of the present invention are capable of expressing short RNA sequences of choice as circular RNAs.

In one embodiment of the present invention is the size of the circular RNAs 25-200 nucleotides, such as 40-100 nucleotides, such as 50-70 nucleotides.

As mentioned above are these RNA sequences in one embodiment of the present invention microRNAs or anti-miRs.

In another embodiment of the invention are these RNA sequences the HIV Rev Response Element (RRE) or other viral RNAs.

In one embodiment of the present invention the circular RNA comprises (streptavidin) aptamer sequences.

In one embodiment of the present invention the circular RNA comprises motifs for RNA binding proteins.

In one embodiment of the present invention the RNA produced from DNAs, vector or cassettes comprises motifs facilitating RNA-RNA interaction.

### Splicing

Several methods of RNA splicing occur in nature; the type of splicing depends on the structure of the spliced sequence and the catalysts required for splicing to occur.

A splice donor site (SD, the 5′ end of the sequence) and an acceptor site (SA, 3′ end of the sequence) are usually required for splicing.

The splice donor site (SD) includes an almost invariant sequence GU at the 5′ end of the polynucleotide, within a larger, less highly conserved region.

The splice acceptor site (SA) at the 3′ end of the polynucleotide terminates the intronic polynucleotide with an almost invariant AG sequence.

Thus, in one embodiment of the present invention the RNA polynucleotide of the present invention comprises a splice acceptor site (SA) and a splice donor site (SD).

Another embodiment of the present invention relates to a single or double stranded DNA molecule of the present invention that comprises a splice acceptor site (SA) and a splice donor site (SD).

Upstream (5′-ward) from the AG there is usually a region high in pyrimidines (C and U), or polypyrimidine tract.

Upstream from the polypyrimidine tract is the branch point, which usually includes an adenine nucleotide.

In further embodiment of the present invention the RNA polynucleotide comprises a region high in pyrimidines (C and U), or polypyrimidine tract.

The splicing of the two ends of the polynucleotide may also be done by self-splicing which occurs through the function of a ribozyme that performs the functions of the spliceosome by RNA alone.

Thus, in one embodiment of the present invention comprises the polynucleotide of the present invention a ribozyme function that allows splicing of the two ends.

In another embodiment of the present invention is the splicing done by ribonucleases that cleave the RNA and ligases join the ends together.

In another embodiment of the present invention is the splicing done by an inverted group I intron.

In another embodiment of the present invention comprises the polynucleotide of the present invention a cis-acting regulatory site i.e. silencers and/or enhancers.

Splicing enhancers are sites to which splicing activator proteins bind, increasing the probability that a nearby site will be used as a splice junction.

These also may occur in the intron (intronic splicing enhancers, ISE) or exon (exonic splicing enhancers, ESE).

Most of the activator proteins that bind to ISEs and ESEs are members of the SR protein family. Such proteins contain RNA recognition motifs and arginine and serine-rich (RS) domains.

Splicing silencers are sites to which splicing repressor proteins bind, reducing the probability that a nearby site will be used as a splice junction.

These can be located in the intron itself (intronic splicing silencers, ISS) or in a neighboring exon (exonic splicing silencers, ESS). They vary in sequence, as well as in the types of proteins that bind to them.

The majority of splicing repressors are heterogeneous nuclear ribonucleoproteins (hnRNPs) such as hnRNPA1 and polypyrimidine tract binding protein (PTB).

Spliceosomal splicing and self-splicing involves a two-step biochemical process.

Both steps involve transesterification reactions that occur between RNA nucleotides.

Spliceosomal and self-splicing transesterification reactions occur via two sequential transesterification reactions.

First, the 2′OH of a specific branch-point nucleotide within the intron that is defined during spliceosome assembly performs a nucleophilic attack on the first nucleotide of the intron at the 5′ splice site forming the lariat intermediate.

Second, the 3′OH of the released 5′ exon then performs a nucleophilic attack at the last nucleotide of the intron at the 3′ splice site thus joining the exons and releasing the intron lariat.

In one embodiment of the present invention is the splicing of the two ends of the polynucleotide done in vitro.

In another embodiment of the present invention is the splicing of the two ends of the polynucleotide done in vivo e.g. where the polynucleotide is expressed and subsequently made circular through the function of the host splice machinery.

In another embodiment of the present invention is the splicing of the two ends of the polynucleotide regulated by introducing splice-enhancer or splice-repressor sequences.

### Promoters

A promoter is a region of DNA that initiates transcription of a particular gene.

Promoters are usually located near the genes they transcribe, on the same strand and upstream (towards the 3′ region of the anti-sense strand).

For the transcription to take place the RNA polymerase must attach to the DNA near a gene.

Promoters contain specific DNA sequences and response elements that provide a secure initial binding site for RNA polymerase and for transcription factors that recruit RNA polymerase.

These transcription factors have specific activator or repressor sequences of corresponding nucleotides that attach to specific promoters and regulate gene expressions.

The core promoter is the minimal portion of the promoter required to properly initiate transcription which includes a Transcription Start Site (TSS) and elements directly upstream.

These elements are a binding site for a RNA polymerase and general transcription factor binding sites, e.g. a TATA box.

Thus, in one embodiment of the present invention is the promoter a core promoter.

A proximal promoter is the proximal sequence upstream of the gene that tends to contain primary regulatory elements.

These elements are usually approximately 250 bp upstream of the start site and they include specific transcription factor binding sites.

A distal promoter is the distal sequence upstream of the gene that may contain additional regulatory elements, often with a weaker influence than the proximal promoter.

These element are usually anything further upstream (but not an enhancer or other regulatory region whose influence is positional/orientation independent) and specific transcription factor binding sites.

Thus, in a further embodiment of the present invention comprises the promoter a proximal and/or a distal promoter.

In another embodiment of the present invention is the promoter a cell or tissue-specific promoter that allows for expression in a desired type of cell or tissue.

### Artificial Nucleic Acid Sequences and Modifications

The nucleic acid sequences may be produced synthetically or transcribed in vitro from an expression vector.

Such nucleic acid sequences may comprise, by direct incorporation or subsequent modification, alternative nucleobase units or modifications.

Thus, one embodiment of the present invention relates to the RNA polynucleotide of the present invention, wherein at least one of the nucleobase units of the polynucleotide is a Locked Nucleic Acid (LNA) nucleobase unit, a INA unit, a TINA unit, a 2′-OMe RNA unit and/or a 2′-fluoro DNA unit.

### Cells

The nucleic acids of the present invention may be comprised within a cell.

In one embodiment, the cell, or in another embodiment, cell systems of this invention comprise primary cultures or other.

“Primary cultures” refers, in one embodiment, to a culture of cells that is directly derived from cells or tissues from an individual, as well as cells derived by passage from these cells, or immortalized cells.

In one embodiment, “cell line” refers to a population of cells capable of continuous or prolonged growth and division in vitro.

The term “cell lines” also includes immortalized cells.

Often, cell lines are clonal populations derived from a single progenitor cell. Such cell lines are also termed “cell clones”.

It is further known in the art that spontaneous or induced changes can occur in karyotype during storage or transfer of such clonal populations.

Therefore, cells derived from the cell clones referred to may not be precisely identical to the ancestral cells or cultures.

According to the present invention, such cell clones may be capable of supporting replication of a vector, virus, viral particle, etc., of this invention, without a significant decrease in their growth properties, and are to be considered as part of this invention.

Thus relates one embodiment of the present invention to a cell comprising the nucleic acid according to the present invention, the composition of the present invention or the expression or cassette vector of the present invention.

It is to be understood that the nucleic acid of the present invention relates to the RNA polynucleotide of the invention or the DNA of the present invention e.g. a vector or expression cassette.

### Pharmaceutical Compositions

One embodiment of the present invention relates to a composition comprising a nucleic acid molecule according to the invention suspended in a suitable amount of a pharmaceutical acceptable diluent or excipient.

In another embodiment, this invention provides for compositions comprising an isolated nucleic acid, vector or cell of this invention, or an isolated nucleic acid.

In one embodiment, the term “composition” refers to any such composition suitable for administration to a subject, and such compositions may comprise a pharmaceutically acceptable carrier or diluent, for any of the indications or modes of administration as described. The active materials in the compositions of this invention can be administered by any appropriate route, for example, orally, parenterally, intravenously, intradermally, subcutaneously, or topically, in liquid or solid form.

Such composition is usually known as a pharmaceutical composition.

Thus, one embodiment of the present invention relates to a pharmaceutical composition comprising a polynucleotide of the present invention, a pharmaceutically acceptable diluent, carrier, or adjuvant, wherein the circular polynucleotide is at least partly complementary to a human microRNA sequence or comprises a guide sequence corresponding to the seed sequence of a human microRNA sequence.

It is to be understood that any applicable drug delivery system may be used with the compositions and/or agents/vectors/cells/nucleic acids of this invention, for administration to a subject, and is to be considered as part of this invention.

It is also to be understood that any of the methods of this invention, whereby a nucleic acid, vector or cell of this invention is used, may also employ a composition comprising the same as herein described, and is to be considered as part of this invention.

“Pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans.

The term “excipient” refers to a diluent, adjuvant, carrier, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The term “adjuvant” refers to a compound or mixture that enhances the immune response to an antigen. An adjuvant can serve as a tissue depot that slowly releases the antigen and also as a lymphoid system activator that non-specifically enhances the immune response. Often, a primary challenge with an antigen alone, in the absence of an adjuvant, will fail to elicit a humoral or cellular immune response.

Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvants such as BCG (bacille Calmette-Guerin) and Corynebacterium parvmm.

Preferably, the adjuvant is pharmaceutically acceptable.

Thus relates one embodiment of the present invention to a composition comprising a nucleic acid molecule according to the present invention suspended in a suitable amount of a pharmaceutical acceptable diluent or excipient.

Methods of Inhibiting microRNAs and Treatment

The polynucleotides and the expression vectors of the present invention can be used to regulate the function of microRNAs and thereby also the expression of specific genes.

Thus, one aspect and an embodiment of the present invention relates to a method for reducing the effective amount of a microRNA or microRNA target in a cell or an organism, comprising administering a pharmaceutical composition of the present invention, to the cell or the organism.

An embodiment of the present invention relates to a method of the present invention, wherein the reduction of the effective amount of the microRNA or micro RNA target occurs via in vivo antagonism.

A further aspect and an embodiment of the present invention relates to a method for de-repression of a target RNA of a microRNA in a cell or an organism, comprising administering a pharmaceutical composition of the present invention, to the cell or the organism.

Another aspect and an embodiment of the present invention relates to the use of a polynucleotide circular RNA of the present invention for the manufacture of a medicament for the treatment of a disease or medical disorder associated with the presence or over-expression of the microRNA.

Another aspect and an embodiment of the present invention relates to a method for the treatment of a disease or medical disorder associated with the presence or over-expression of the microRNA, comprising the step of administering a pharmaceutical composition of the present invention to a person in need of treatment.

Another aspect and an embodiment of the present invention relates to the use of a polynucleotide circular RNA of the present invention for the manufacture of a medicament for the treatment of a disease or medical disorder associated with the presence or over-expression of the other RNA species or RNA-binding proteins.

In one embodiment of the present invention is the disease selected from the group consisting of cancer, a viral infection, obesity, diabetes, and a neurological disease.

In another embodiment of the present invention is the disease selected form the group consisting of Parkinson's disease, lymphoma, leukemia, CLL, colon cancer, breast cancer, heart disease, schizophrenia, HCV, herpesvirus and HIV.

It should be noted that embodiments and features described in the context of one of the aspects of the present invention also apply to the other aspects and embodiments of the invention.

All patent and non-patent references cited in the present application, are hereby incorporated by reference in their entirety.

In yet another embodiment the present invention can be used to cure or ameliorate diseases such as but not limited to those listed below.

miR2Disease, a manually curated database, aims at providing a comprehensive resource of miRNA deregulation in various human diseases. Each entry in the miR2Disease contains detailed information on a miRNA-disease relationship, including miRNA ID, disease name, a brief description of the miRNA-disease relationship, miRNA expression pattern in the disease state, detection method for miRNA expression, experimentally verified miRNA target gene(s), and literature reference. DOID: Disease Ontology ID. http://www.mir2disease.org/Abdominal
Aortic Aneurysm DOID:7693
acute lymphoblastic leukemia (ALL) DOID:9952
acute myeloid leukemia (AML) DOID:9119
acute myocardial infarction DOID:9408
acute promyelocytic leukemia (APL) DOID:9119
adenoma DOID:657
adrenocortical carcinoma DOID:3948
alcoholic liver disease (ALD) DOID:9454
Alzheimer's disease DOID:10652
anaplastic thyroid carcinoma (ATC) DOID:7012
anxiety disorder DOID:2030
asthma DOID:2841
Astrocytoma DOID:3069
Atopic Dermatitis DOID:3310
autism spectrum disorder (ASD) DOID:1208
B-cell chronic lymphocytic leukemia DOID:1040
B-cell lymphoma DOID:353
Becker muscular dystrophy (BMD) DOID:14691
bladder cancer DOID:4007
Brain Neoplasm DOID:1319
breast cancer DOID:3459
Burkitt lymphoma DOID:8584
cardiac hypertrophy DOID:11984
cardiomyopathy DOID:3978
Cardiovascular Disease DOID:2348
Cerebellar neurodegeneration DOID:2786
cervical cancer DOID:2893
cholangiocarcinoma DOID:4947
cholesteatoma DOID:869
choriocarcinoma DOID:3594
chronic lymphocytic leukemia (CLL) DOID:1040
chronic myeloid leukemia (CML) DOID:8552
chronic pancreatitis DOID:13315
Colon Carcinoma DOID:1520
colorectal cancer DOID:1985
congenital heart disease DOID:114
coronary artery disease DOID:3363
Cowden Syndrome DOID:3471
dermatomyositis (DM) DOID:10223
Diabetic Nephropathy DOID:2370
diarrhea predominant irritable bowel syndrome (IBS-D) DOID:9778
diffuse large B-cell lymphoma (DLBCL) DOID:353
Dilated Cardiomyopathy DOID:12930
Down syndrome (DS) DOID:14250
Duchenne muscular dystrophy (DMD) DOID:14691
endometrial cancer DOID:2871
Endometrial Endometrioid Adenocarcinoma DOID:5827
Endometriosis DOID:289
epithelial ovarian cancer (EOC) DOID:4001
esophageal cancer DOID:1107
Esophagus squamous cell carcinoma DOID:3748
essential thrombocythemia (ET) DOID:2224
facioscapulohumeral muscular dystrophy (FSHD) DOID:11727
follicular lymphoma (FL) DOID:8524
follicular thyroid carcinoma (FTC) DOID:3962
frontotemporal dementia DOID:10216
gastric cancer (stomach cancer) DOID:5517
glioblastoma DOID:3068
glioblastoma multiforme (GBM) DOID:3073
glioma DOID:2627
glomerular disease DOID:2976
Glomerulosclerosis DOID:3945
hamartoma DOID:3462
HBV-related cirrhosis DOID:5082
HCV infection DOID:7324
head and neck cancer DOID:11934
head and neck squamous cell carcinoma (HNSCC) DOID:5520
hearing loss DOID:10004
Heart Disease DOID:114
heart failure DOID:6000
Hepatitis B DOID:2043
Hepatitis C DOID:1883
hepatocellular carcinoma (HCC) DOID:684
Hilar Cholangiocarcinoma DOID:4927
Hodgkin's lymphoma DOID:8567
homozygous sickle cell disease (HbSS) DOID:10923
Huntington's disease (HD) DOID:12858
Hypertension DOID:10763
Hypopharyngeal cancer DOID:8532
Inclusion body myositis (IBM) DOID:3429
Insulinoma DOID:3903
Intrahepatic cholangiocarcinoma (ICC) DOID:4928
kidney cancer DOID:4451
Kidney Disease DOID:557
laryngeal carcinoma DOID:6624
Late Insomnia (sleep disease DOID:535) DOID:535
Leiomyoma of lung DOID:5136
Leukemia DOID:1240
limb-girdle muscular dystrophies types 2A (LGMD2A) DOID:11724
lipoma DOID:9291
Lung Adenocarcinoma DOID:3910
lung cancer DOID:3905
lymphoproliferative disease DOID:619
malignant lymphoma DOID:353
malignant melanoma DOID:1909
Malignant mesothelioma (MM) DOID:1790
mantle cell lymphoma (MCL) DOID:353
medulloblastoma DOID:3858
melanoma DOID:1909
meningioma DOID:3565
metabolic disease DOID:14667
miyoshi myopathy (MM) DOID:423
multiple myeloma (MM) DOID:9538
multiple sclerosis DOID:2377
MYC-rearranged lymphoma DOID:353
Myelodysplastic Syndrome DOID:4972
myeloproliferative disorder DOID:2356
myocardial infarction DOID:5844
myocardial injury DOID:12931
myoma DOID:2691
nasopharyngeal carcinoma (NPC) DOID:9261
nemaline myopathy (NM) DOID:3191
Nephritis DOID:10952
neuroblastoma (NB) DOID:769
neutrophilia is_obsolete
Niemann-Pick type C (NPC) disease DOID:14770
non-alcoholic fatty liver disease (NAFLD) DOID:46
non-small cell lung cancer (NSCLC) DOID:3908
Obesity DOID:9970
Oesophageal adenocarcinoma DOID:4914
Oral Carcinoma DOID:8894
osteosarcoma DOID:3347
ovarian cancer (OC) DOID:2144
pancreatic cancer DOID:4905
pancreatic ductal adenocarcinoma (PDAC) DOID:3498
Pancreatic Neoplasia DOID:3588
Panic Disease DOID:594
papillary thyroid carcinoma (PTC) DOID:3969
Parkinson's disease DOID:14330
PFV-1 infection DOID:934
Pharyngeal Disease DOID:939
pituitary adenoma DOID:3829
Polycystic Kidney Disease DOID:5898
Polycystic liver disease DOID:1173
polycythemia vera (PV) is_obsolete
polymyositis (PM) DOID:10222
primary biliary cirrhosis (PBC) DOID:12236
primary myelofibrosis DOID:5737
prion disease DOID:649
prostate cancer DOID:10286
Psoriasic Arthritis DOID:9008
psoriasis DOID:9088
pulmonary hypertension DOID:6432
recurrent ovarian cancer DOID:6708
Renal Cell Carcinoma DOID:4450
renal clear cell carcinoma DOID:4467
retinitis pigmentosa (RP) DOID:10584
retinoblastoma DOID:768
rhabdomyosarcoma DOID:3247
Rheumatic Heart Disease DOID:9814 and Atrial Fibrillation DOID:9814
Rheumatoid Arthritis DOID:7148
sarcoma DOID:1115
schizophrenia DOID:5419
sepsis DOID:2292
serous ovarian cancer DOID:5745
Sezary syndrome DOID:8541
skin disease DOID:37
Small Cell Lung Cancer DOID:3907
Spinocerebellar ataxia DOID:1441
squamous carcinoma DOID:1749
T-cell leukemia DOID:715
teratocarcinoma DOID:3305
testicular germ cell tumor DOID:5556
thalassemia DOID:10241
thyroid cancer DOID:3963
tongue squamous cell carcinoma DOID:10944
tourette's syndrome DOID:11119
type 2 diabetes DOID:9351
ulcerative colitis (UC) DOID:8577
uterine leiomyoma (ULM) DOID:13223
uveal melanoma DOID:1909
vascular disease DOID:178
vesicular stomatitis DOID:10881
Waldenstrom Macroglobulinemia (WM) DOID:9080

Other diseases are metabolic diseases in general, cancer diseases and regulation of insulin and blood sugar.

### Especially Interesting Disease Target Comprise

Inhibition of onco-miRs, such as miR-15, miR16, miR-17, miR-18, miR-19, miR-21, miR-155 or miR-191, as a potential cancer treatment.

Inhibition of miR-9 and miR-128 known to be upregulated in Alzheimers disease, as a potential treatment.

Inhibition of miR-29 involved in insulin resistance as a potential treatment of type 2 diabetes.

Inhibition of miR-208a known to induce arrhythmia as a potential treatment of cardiac hypertrophy.

Inhibition of virally encoded miRNAs, such hcmv-miR-UL112, to potentially treat viral infection.

Treatment of hepatits C by inhibition of miR-122 known to be essential for Hepatitis C infection.

### Other Circular RNA as RNA/Motif Blocker are:

Blocking specific target sites for onco-miRs, such as miR-17:E2F1, miR-19:PTEN, miR-21:PDCD4, or miR-155:SOCS1, as a potential cancer treatment.

Blocking the Open-Reading-Frame (ORF) of oncogenes, such as Myc or Ras. This approach also applies to the potential treatment of other diseases with gain-of-function gene expression.

### Circular RNA as Protein Decoys:

Sequestering and inhibition of RNA-binding proteins involved in cancer progression, such as Sam68 or SF2/ASF, as a potential cancer treatment.

Sequestering and inhibition of RNA-binding proteins involved in neurological degeneration, such as Nova or Hu, as a potential treatment.

Sequestering and inhibition of virally encoded RNA-binding proteins, such as Rev, as a potential anti-viral treatment.

### Additional Aspects and Embodiments

Additional aspects and embodiments of the invention are provided below.

According to an aspect and an embodiment, the present invention concerns a circular RNA polynucleotide which is at least partly complementary to a human or animal microRNA sequence.

The expression “circular RNA polynucleotide” refers to RNA polynucleotide consisting of a circular structure as well as a structure comprising a circular structure, such as a circular lariat.

According to an embodiment, the invention concerns circular RNA polynucleotide having at least one mismatch to said microRNA. A preferred target sequence in the circular RNA has at least one mismatch to the microRNA that makes it resistant to endonucleolytic cleavage. Accordingly, preferably said target sequence is not fully complementary to a non-native or wild-type sequence, but comprises a number of mutations selected among 1, 2, 3, 4, 5, 6, 7, 8, 9, or 10 mutations, insertions and/or deletions.

According to an embodiment, the invention concerns circular RNA polynucleotide, wherein said circular RNA polynucleotide is resistant to endonucleolytic cleavage caused by said microRNA. A preferred sequence is resistant to endonucleolytic cleavage by the microRNA it regulate as interpreted from a simple assay where both the circular RNA and microRNA is expressed in the same cell. The assay, demonstrated below microRNA-7, can be conducted by a skilled scientist for any other part of circular RNA and microRNA. As an example of a micro-RNA, that does not contain any mismatches in the central part and therefore is able to cleave the circular RNA, microRNA-671 included.

A circular RNA polynucleotide according to the invention preferably allows inhibition of interactions between microRNA and their RNA target. A circular RNA polynucleotide according to the invention is preferably resistant to degradation by exonucleases or endonucleases.

According to an embodiment, the invention concerns circular RNA polynucleotide, wherein said circular RNA polynucleotide contains more than one binding site for microRNA. The microRNA may be any microRNA.

According to an embodiment, the invention concerns circular RNA polynucleotide, wherein said circular RNA polynucleotide contains at least two, such as three, at least four, such as five, at least six, such as seven, at least eight, such as nine binding sites for microRNA.

According to an embodiment, the invention concerns circular RNA polynucleotide, wherein said circular RNA polynucleotide contains at least ten, such as fifteen, at least twenty, such as twenty five, at least thirty, such as thirty five binding sites for microRNA.

According to an embodiment, the invention concerns circular RNA polynucleotide, for use as a medicament.

According to an embodiment, the invention concerns circular RNA polynucleotide, for use in vivo or in vitro in laboratory work.

According to an embodiment, the invention concerns circular RNA polynucleotide, for use in vivo or in vitro in laboratory work such as but not limited to inhibition of miRNAs, such as but not limited to miR-15, miR-16, miR-17, in cell cultures or organisms to study the relevance/loss-of function phenotype of the specific miRNA. Such uses comprise, but are not limited to:

Inhibition of miRNA target sites, such as but not limited to the miR-17 target site in E2F1 mRNA or the miR-19 target site in PTEN mRNA, in cell cultures or organisms to study the relevance of the specific target site.

Inhibition of protein binding sites, such as but not limited to the HuR binding site in the TNF-α mRNA or the Pumilio (Pum1 and Pum2) binding site in the E2F3 mRNA, in cell cultures or organisms to study the relevance of the specific protein-mRNA interaction.

Inhibition of protein binding, protein activity or protein function, such as but not limited to the uPAR binding of uPA or the activity of HCV NS5B RNA replicase, in cell cultures or organisms to study the effect of protein loss-of-function.

Inhibition of translation of mRNAs, such as but not limited to RAS and MYC, in cell cultures or organisms to study the effect of the specific translation and to deplete the specific protein.

Inhibition of the secondary structure of RNAs, such as but not limited to tRNA, snoRNA, pre-miRNA, in cell cultures or organisms to study the relevance of the secondary structure.

Inhibition of DNA motifs, such as but not limited to TATA-boxes, GC-islands and transcription factor binding sites, in cell cultures or organisms to study the effect of the specific DNA motifs.

Coupling of circular RNA with fatty acid moieties to increate transfection efficiency and to tether fatty acid binding proteins in cell cultures and organisms.

Coupling of circular RNA with carbohydrate moieties, oligo- and polysaccharides, such as but not limited to galactose, glycogen, or poly-L-Lysine to tether carbohydrate binding proteins in cell cultures and organisms.

Coupling of circular RNA with protein/peptide, moieties, such as NPY (NeuroPeptide Y), Erythropoietin, or Follistatin to tether protein-binding proteins in cell cultures and organisms.

Coupling of circular RNA with fluorescent dyes, such as but not limited to cy3 and cy5, to track the presence of circular RNA in cell cultures and organisms.

Coupling of circular RNA with other chemical structures, such as but not limited to NADH, aspirin, or aspartame to potentially study the interactome of the coupled chemical structure.

The circular RNA can contain natural and/or unnatural nucleotides, such as but not limited to -2′OMe, -2′F, or LNA, to modify the chemical properties of the circular RNA.

### Regulation of Circular RNA Expression:

Circular RNA can be introduced into cells, tissues or whole animals using either in vitro synthesized circular RNA or circular RNA expressing cassettes, such as but not limited to plasmids or retroviral vectors using delivery methods such as but not limited to transfection or electroporation.

Control of circular RNA expression in cells, tissue and whole animal by using inducible promoters, such as but not limited to Tet-on or LTR-Tat, in cells, tissue and whole animal.

Expression of RNA circles from tissue specific promoters to allow tissue specific inhibition of RNA and protein in cells, tissue and whole animal.

Control of circular RNA expression using recombination-based systems, such as but not limited to Cre-Lox or Flp-In in cells, tissue and whole animal.

Control of circular RNA expression using splicing regulators, such as but not limited to hnRNP-A1, PTB, or spliceostatin A.

Control of circular RNA expression using RNA binding proteins, such as HuR or RNase H.

Control of circular RNA expression using RNA depletion methods, such as but not limited to siRNAs, miRNAs and Gapmers.

Control of circular RNA expression using antisense technology, such as but not limited to antisense Morpholino.

According to an embodiment, the invention concerns circular RNA polynucleotide, which comprises a splice acceptor site (SA) and a splice donor site (SD).

According to an embodiment, the invention concerns circular RNA polynucleotide, wherein the polynucleotide comprises a region of contiguous nucleobase sequence which is 100% complementary to the human or animal microRNA seed region.

According to an embodiment, the invention concerns circular RNA polynucleotide, wherein the polynucleotide comprises a region of contiguous nucleobase sequence which is partially complementary to the human or animal microRNA seed region.

According to an embodiment, the invention concerns circular RNA polynucleotide, wherein at least part of the polynucleotide is complementary to the mature human or animal microRNA sequence.

According to an embodiment, the invention concerns circular RNA polynucleotide comprising an antisense sequence that comprises a guide sequence corresponding to the seed sequence of microRNA.

According to an aspect and an embodiment, the present invention concerns DNA comprising:
a) a promoter,
b) a nucleic acid sequence that is completely or partially complementary to a human or animal microRNA sequence,
c) a splice acceptor site (SA) upstream or downstream of said nucleic acid sequence of b), and
d) a splice donor site (SD) downstream or upstream of said nucleic acid sequence of b).

According to an aspect and an embodiment, the present invention concerns DNA comprising:
a) a promoter,
b) a nucleic acid sequence that is complementary to the seed sequence of a human or animal microRNA sequence,
c) a splice acceptor site (SA) upstream or downstream of said nucleic acid sequence of b), and
d) a splice donor site (SD) downstream or upstream of said nucleic acid sequence of b).

According to an aspect and an embodiment, the present invention concerns DNA comprising:
a) a promoter,
b) two or more nucleic acid sequences that is selected from the group consisting of a nucleic acid sequence that is completely or partially complementary to a human or animal microRNA sequence and a nucleic acid sequence that is complementary to the seed sequence of a human or animal microRNA sequence,
c) a splice acceptor site (SA) upstream or downstream of said two or more nucleic acid sequences of b), and
d) a splice donor site (SD) downstream or upstream of said two or more nucleic acid sequences of b).

According to an embodiment, the invention concerns DNA, which further comprises inverted repeats.

According to an embodiment, the invention concerns DNA, wherein said nucleic acid sequence(s) is not microRNA or does not comprise a sequence corresponding to the complete sequence of microRNA.

According to an aspect and an embodiment, the present invention concerns RNA transcribed from the DNA according to the invention.

According to an embodiment, the invention concerns RNA polynucleotide, wherein at least one of the nucleobase units of the polynucleotide is selected among a Locked Nucleic Acid (LNA) unit, a Unlocked Nucleic Acid (UNA) unit, a TINA unit, a 2′-OMe RNA unit and a 2′-fluoro RNA unit.

According to an aspect and an embodiment, the present invention concerns a cell comprising the circular RNA polynucleotide according to the invention.

According to an aspect and an embodiment, the present invention concerns a pharmaceutical composition comprising a circular polynucleotide according to the invention, and at least one of a pharmaceutically acceptable diluent, carrier and adjuvant, wherein the circular polynucleotide is at least partly complementary to a human or animal microRNA sequence or comprises a guide sequence complementary to the seed sequence of a human or animal microRNA sequence.

According to an aspect and an embodiment, the present invention concerns a method for inhibiting, inactivating or reducing the effective amount of microRNA in a cell or an organism, comprising administering to the cell or the organism an entity selected among the group consisting of a circular RNA polynucleotide, DNA, RNA, a cell and a pharmaceutical composition according to the invention.

According to an embodiment, the present invention concerns the method wherein the reduction of the effective amount of the microRNA occurs via in vivo antagonism.

According to an aspect and an embodiment, the present invention concerns a method for de-repression of a target mRNA of a microRNA in a cell or an organism, comprising administering to the cell or the organism an entity selected among the group consisting of a circular RNA polynucleotide, DNA, RNA, a cell and a pharmaceutical composition according to the invention.

According to an aspect and an embodiment, the present invention concerns a use of a circular RNA polynucleotide according to the invention for the manufacture of a medicament for the treatment, amelioration or prevention of a disease or medical disorder associated with the presence or over-expression of microRNA.

A circular RNA polynucleotide according to the invention for the treatment, amelioration or prevention of a disease or medical disorder associated with the presence or over-expression of microRNA.

According to an aspect and an embodiment, the present invention concerns A method for the treatment, amelioration or prevention of a disease or medical disorder associated with the presence or over-expression of microRNA, comprising the step of administering an entity selected among the group consisting of a circular RNA polynucleotide, DNA, RNA, a cell and a pharmaceutical composition according to the invention, to a person or animal in need of treatment.

According to an aspect and an embodiment, the present invention concerns the use, circular RNA polynucleotide, or method according to the invention, wherein the disease or medical disorder is selected from the group consisting of Parkinson's disease, cancer, a viral infection, Hepatitis C, diabetes, epilepsy, a neurological disease, insulin deficiency and blood sugar, and the following diseases and medical disorders: Abdominal Aortic Aneurysm, acute lymphoblastic leukemia (ALL), acute myeloid leukemia (AML), acute myocardial infarction, acute promyelocytic leukemia (APL), adenoma, adrenocortical carcinoma, alcoholic liver disease (ALD), Alzheimer's disease, anaplastic thyroid carcinoma (ATC), anxiety disorder, asthma, Astrocytoma, Atopic Dermatitis, autism spectrum disorder (ASD), B-cell chronic lymphocytic leukemia, B-cell lymphoma, Becker muscular dystrophy (BMD), bladder cancer, Brain Neoplasm, breast cancer, Burkitt lymphoma, cardiac hypertrophy, cardiomyopathy, Cardiovascular Disease, Cerebellar neurodegeneration, cervical cancer, cholangiocarcinoma, cholesteatoma, choriocarcinoma, chronic lymphocytic leukemia (CLL), chronic myeloid leukemia (CML), chronic pancreatitis, Colon Carcinoma, colorectal cancer, congenital heart disease, coronary artery disease, Cowden Syndrome, dermatomyositis (DM), Diabetic Nephropathy, diarrhea predominant irritable bowel syndrome (IBS-D), diffuse large B-cell lymphoma (DLBCL), Dilated Cardiomyopathy, Down syndrome (DS), Duchenne muscular dystrophy (DMD), endometrial cancer, Endometrial Endometrioid Adenocarcinoma, Endometriosis, epithelial ovarian cancer (EOC), esophageal cancer, Esophagus squamous cell carcinoma, essential thrombocythemia (ET), facioscapulohumeral muscular dystrophy (FSHD), follicular lymphoma (FL), follicular thyroid carcinoma (FTC), frontotemporal dementia, gastric cancer (stomach cancer), glioblastoma, glioblastoma multiforme (GBM), glioma, glomerular disease, Glomerulosclerosis, hamartoma, HBV-related cirrhosis, HCV infection, head and neck cancer, head and neck squamous cell carcinoma (HNSCC), hearing loss, Heart Disease, heart failure, Hepatitis B, Hepatitis C, hepatocellular carcinoma (HCC), Hilar Cholangiocarcinoma, Hodgkin's lymphoma, homozygous sickle cell disease (HbSS), Huntington's disease (HD), Hypertension, Hypopharyngeal cancer, Inclusion body myositis (IBM), Insulinoma, Intrahepatic cholangiocarcinoma (ICC), kidney cancer, Kidney Disease, laryngeal carcinoma, Late Insomnia (sleep disease), Leiomyoma of lung, Leukemia, limb-girdle muscular dystrophies types 2A (LGMD2A), lipoma, Lung Adenocarcinoma, lung cancer, lymphoproliferative disease, malignant lymphoma, malignant melanoma, Malignant mesothelioma (MM), mantle cell lymphoma (MCL), medulloblastoma, melanoma, meningioma, metabolic disease, miyoshi myopathy (MM), multiple myeloma (MM), multiple sclerosis, MYC-rearranged lymphoma, Myelodysplastic Syndrome, myeloproliferative disorder, myocardial infarction, myocardial injury, myoma, nasopharyngeal carcinoma (NPC), nemaline myopathy (NM), Nephritis, neuroblastoma (NB), neutrophilia, Niemann-Pick type C (NPC) disease, non-alcoholic fatty liver disease (NAFLD), non-small cell lung cancer (NSCLC), Obesity, Oesophageal adenocarcinoma, Oral Carcinoma, osteosarcoma, ovarian cancer (OC), pancreatic cancer, pancreatic ductal adenocarcinoma (PDAC), Pancreatic Neoplasia, Panic Disease, papillary thyroid carcinoma (PTC), Parkinson's disease, PFV-1 infection, Pharyngeal Disease, pituitary adenoma, Polycystic Kidney Disease, Polycystic liver disease, polycythemia vera (PV), polymyositis (PM), primary biliary cirrhosis (PBC), primary myelofibrosis, prion disease, prostate cancer, Psoriasic Arthritis, psoriasis, pulmonary hypertension, recurrent ovarian cancer, Renal Cell Carcinoma, renal clear cell carcinoma, retinitis pigmentosa (RP), retinoblastoma, rhabdomyosarcoma, Rheumatic Heart Disease and Atrial Fibrillation, Rheumatoid Arthritis, sarcoma, schizophrenia, sepsis, serous ovarian cancer, Sezary syndrome, skin disease, Small Cell Lung Cancer, Spinocerebellar ataxia, squamous carcinoma, T-cell leukemia, teratocarcinoma, testicular germ cell tumor, thalassemia, thyroid cancer, tongue squamous cell carcinoma, tourette's syndrome, type 2 diabetes, ulcerative colitis (UC), uterine leiomyoma (ULM), uveal melanoma, vascular disease, vesicular stomatitis, and Waldenstrom Macroglobulinemia (WM).

According to an aspect and an embodiment, the present invention concerns use of a polynucleotide according to the invention for recruitment and inhibition of RNA-binding proteins. This is also referred to as protein decoy.

According to an aspect and an embodiment, the present invention concerns use of a polynucleotide according to the invention for blocking and inhibition of RNA. According to an embodiment, the invention concerns the use, wherein said RNA is selected among miRNA target sites, open-reading-frames and non-coding RNA.

According to an aspect and an embodiment, the present invention concerns use of a polynucleotide according to the invention for assembly of proteins into new functional entities.

According to an aspect and an embodiment, the present invention concerns a method for expression of a polynucleotide according to the invention in a target cell using a viral vector.

According to an aspect and an embodiment, the present invention concerns a method for expression of a polynucleotide according to the invention, wherein said viral vector is selected among an adenoviral, retroviral, baculoviral sindbis derived vector, polio virus derived vector and lentiviral vector.

According to an aspect and an embodiment, the present invention concerns a method for expression of a polynucleotide according to the invention in a target cell using DNA.

According to an aspect and an embodiment, the present invention concerns a method for expression of a polynucleotide according to the invention, wherein said DNA is selected among plasmids or linear DNA vectors.

According to an aspect and an embodiment, the present invention concerns a method for delivery of a a polynucleotide according to the invention by a method selected among chemical-based transfection, non-chemical methods, particle-based methods, nucleofection-based methods, heat-shock-based methods, homologous DNA recombination, CRISPR system, flip-in system, and Cre-lox system.

The delivery may inter alia be performed by chemical-based transfection, such as but not limited to selected among the calcium phosphate method, liposome-based methods, polymers-based transfection or dendrimers-based transfection; Non-chemical methods, such as but not limited to electroporation, optical transfection, protoplast fusion, or hydrodynamic delivery; Particle-based methods, such as but not limited to magnetofection, impalefection or particle bombardment; Other methods, such as but not limited to nucleofection-based methods or heat-shock-based methods; Stable integration of circular RNA expression vector into host chromosome by, but not limited to, homologous DNA recombination, CRISPR system, flip-in system, and Cre-lox system.

According to an aspect and an embodiment, the present invention concerns a use of circular RNA as a microRNA (miRNA) inhibitor.

The invention will now be described in further details in the following non-limiting examples.

### EXAMPLES

### Example 1

### Natural RNA Circles Function as Efficient miRNA Sponges

microRNAs (miRNAs) are important regulators of gene expression acting by direct base pairing to 3′UTR target sites in mRNAs. Recently, miRNA activity has been shown to be affected by the presence of miRNA sponge transcripts, the so-called competing endogenous RNA (ceRNA) in humans and target mimicry expression in plants.

The present inventors have previously identified a highly expressed circular RNA (cRNA) in human and mouse brain. Here the present inventors show that the cRNA acts as a miR-7 sponge and the present inventors term this circular transcript CiRS-7 (Circular RNA Sponge for miR-7). CiRS-7 harbours more than 70 selectively conserved putative miRNA target sites and it is highly and widely associated with Ago-proteins in a miR-7 dependent manner.

While the circular RNA is completely resistant towards miRNA-mediated target destabilization, it strongly suppresses miR-7 activity resulting in elevated levels of miR-7 targets.

In the mouse brain, we observe overlapping neuronal expression patterns of CiRS-7 and miR-7 in the neocortex and hippocampus, and in thalamus and substantia nigra suggesting a high degree of endogenous interaction.

The present inventors further show that the circular testis specific RNA, SRY, serves as a miR-138 sponge, suggesting that miRNA sponge effects achieved by circular RNA formation is a general phenomenon.

Thus, this serves as the first functional study of a naturally expressed circular RNA.

### Introduction

miRNAs belong to an established class of small non-coding RNA species primarily involved in the post-transcriptional regulation of gene expression. The mature miRNA is incorporated into the RNA induced silencing complex, RISC, and targets specific mRNAs harbouring seed-match target sites in the 3′ UTR.

Currently, the prevalent model suggests that RISC recruits via GW182 a deadenylase complex that in turn facilitates polyA-tail removal thereby destabilizing the targeted mRNA.

While several studies have focused on the regulation of miRNAs, predominantly on the identification of factors involved in the biogenesis of subsets of miRNAs, interesting ‘post-biogenesis’ inhibition of miRNA activity has recently been observed.

Conceptual studies overexpressing miRNA target site concatamers, so-called miRNA sponges, were initially performed and showed to result in loss of miRNA function accompanied with elevated levels of endogenous targets.

Subsequently, an endogenously expressed linear non-coding RNA (ncRNA) was identified as a miRNA sponge, which is able to sequester miRNA-399 using a near perfect, non-cleavable target site.

This phenomenon was coined miRNA mimicry. Very recently, linear competing endogenous RNA (ceRNA) was identified and characterized in mammalian systems emphasizing the existence of pseudogenes and ncRNA expressed to tether and inhibit specific miRNAs.

Collectively, these data suggest that miRNA sponges are widespread regulators of miRNA activity in higher eukaryotes. However, commonly, miRNA sponges or miRNA competing transcripts so far identified all share non-dramatic expression levels and a limited number of miRNA target sites, and it has been questioned whether their expression levels are sufficient for inflicting effective inhibition.

The present inventors recently identified a circular RNA (cRNA) with high endogenous expression levels in human and mouse brain and demonstrated that the cRNA can become endonucleolytically cleaved by miR-671 in an Agog dependent manner, but the function was unknown.

### Results and Discussion

Here the present inventors demonstrate that the cRNA functions as a post-transcriptional regulator of a miRNA implicated in several human disease conditions. Searching for miRNA target sites within the circular RNA, the present inventors identified 73 conventional seed-targets for miR-7.

Plotting the target site position and nucleotide conservation, we observed that almost all identified target sites were selectively and highly conserved in mammals (FIG. 1a).

In addition, analysis of online available Agog HITS-CLIP conducted in mouse brain revealed a high degree of Agog-occupancy on the mouse variant of the circular RNA with sequence reads dispersed throughout the cRNA with highest reads density covering the region with highest 8-mer target sites abundance (FIG. 1b).

A hotspot analysis revealed that the cRNA was one of the most abundant loci in the dataset (FIG. 5, supplementary Table 51). This conforms to our previous observation showing a high expression of cRNA in brain and other studies profiling brain-specific expression of miR-7.

To study the impact of cRNA expression on miR-7 activity, the present inventors established a vector-based system expressing the circular RNA. The biogenesis of circular RNA is currently unknown, however previous studies have shown that exons flanked by inverted repeats induce circular RNA formation in vitro and is suggested to be essential for circular SRY RNA production.

Thus, the present inventors inserted the CiRS-7 exon along with the endogenous flanking sequence (approximately 1000 nt upstream and 200 nt downstream) into pcDNA3 (pcDNA3-CiRS-7-ir, FIG. 1c).

Subsequently, the present inventors copied part of the upstream flanking sequence and inserted it in an inverted orientation downstream (pcDNA3-CiRS-7, FIG. 1c).

Transiently expressing of pcDNA3-CiRS-7, but not the construct lacking the downstream inverted sequence (pcDNA3-CiRS-7-is), resulted in production of cRNA species indistinguishable from the endogenously expressed cRNA by northern analysis (FIG. 1d) and RT-PCR sequencing (data not shown).

In addition, the present inventors constructed a linear and polyadenylated miR-7 sponge containing all miR-7 sponge sites but excluding the splice sites and flanking sequences (CiRS-7-fs, FIG. 1c).

By co-expressing the circular RNA- or the linear RNA-producing vectors with miR-7 or miR-769 expression vectors, the present inventors observed unaffected levels of CiRS-7, whereas the linear counterpart displayed miR-7 sensitivity presumably due to miRNA mediated deadenylation and subsequent degradation (FIG. 1e).

This suggests that CiRS-7 is resistant towards the conventional miRNA destabilization of mRNA and therefore not prone to miR-7 dependent regulation.

To determine whether a CiRS-7 serves as a target for miR-7, the present inventors conducted Ago2-IP in HEK293 cells transiently expressing miR-7 or miR-769 (control) together with myc-tagged Ago2. By qRT-PCR, this showed a high enrichment of endogenous CiRS-7 specifically in miR-7 transfected cells, suggesting that miR-7 indeed is facilitating Ago-association with the CiRS-7 (FIG. 2a).

Furthermore, the present inventors devised a T7 transcript containing all putative miR-7 target sites in conjunction with or without a reported streptavidin-aptamer motif 20.

Incubating the T7 transcripts with lysate from HEK293 cells expressing either miR-7 or miR-769 as control, Ago2 and miR-7 were extracted efficiently only from miR-7 containing cell lysate and in an aptamer specific fashion (FIG. 7, supplementary FIG. 1a-c).

Additionally, using a biotin-coupled miR-7 mimic, the present inventors observed a significant enrichment of CiRS-7 in the miR-7 captured fraction compared to when using biotinylated miR-138 (FIG. 2b).

Thus, the present inventors conclude that the widespread Ago-occupancy is caused by miR-7-directed association of Ago proteins to the prevalent and conserved miR-7 target sites in the cRNA.

To evaluate the subcellular localization of miR-7 and CiRS-7, the present inventors performed RNA-FISH on HEK293 cells (FIG. 2c-d) and HeLa cells (FIG. 8, supplementary FIG. 2b-c) co-transfected with CiRS-7 and miR-7.

This showed a high degree of co-localization between CiRS-7 and miR-7 within distinct cellular bodies. Immunoflourescent staining with the P-body specific marker, hDcp1a21, revealed that CiRS-7 co-localized with P-bodies, however only when co-expressed with miR-7 suggesting that miR-7 compartmentalises CiRS-7 to the bodies (FIG. 2d and FIG. 8, supplementary FIG. 2c).

To investigate whether miR-7 and CiRS-7 are coexpressed at the global scale in the brain, we visualised the expression in the adult mouse brain using an alkaline phosphatase coupled miR-7 and CiRS-7 probe, respectively. The two RNAs showed a distinct neuronal expression (FIG. 2e-h) comparable to GAPDH mRNA (FIG. 9, supplementary FIG. 3a), comprising both cortical pyramidal neurons and interneurons (FIG. 2e-h).

The expression of CiRS-7 in the neuropil areas of the hippocampal CA1 and CA3, and in the molecular layer of the dentate gyrus (FIG. 2e-f), likely reflects that CiRS-7 is translocated into the dendritic tree.

In comparison, miR-7 is expressed in the nucleus and neuronal cell body (FIG. 2g-h, Supplementary FIG. 4a), and notably, also in glial cells in the corpus callosum (FIG. 2f).

Both RNAs are also expressed in thalamus (FIG. 9, supplementary FIG. 3b, CiRS-7 only) and substantia nigra (data not shown). The very high expression of CiRS-7 coinciding with miR-7 expression strongly suggests that miR-7 is under control of CiRS-7 in rodent brain.

The nuclear localisation of miR-7 was confirmed by subcellular fractionation of cells (FIG. 10, supplementary FIG. 4b) which showed a concomitant increase in nuclear prevalence of CiRS-7 when miR-7 was co-expressed (FIG. 10, supplementary FIG. 4b-c).

Moreover, the presence of CiRS-7 resulted in elevated levels of miR-7 (FIG. 10, supplementary FIG. 4c-d), suggesting, collectively, that CiRS-7 and miR-7 have a clear impact on each other in terms of sublocalization within the cell.

To test the effect of CiRS-7 expression on miR-7 activity, the present inventors constructed two luciferase reporter constructs each monitoring miR-7 activity by inserting a perfect miR-7 target (FIG. 3a) or the entire CiRS-7 sequence (FIG. 3b) into the 3′UTR of renilla luciferase (RL).

Co-transfecting the luciferase reporter with miR-7 and CiRS-7 expression vectors showed a substantial reduction of knockdown potential when the CiRS-7 was co-introduced with miR-7 (FIGS. 3a and b).

In fact, the expression of CiRS-7 proved more efficient than a conventional anti-miR approach, suggesting that CiRS-7 is indeed a highly efficient miRNA sponge.

Similarly, using the 3′UTR of EGFR, which is a known target for miR-7, in a luciferase reporter assay, CiRS-7 reduced the knockdown efficiency exerted by miR-7 effectively (FIG. 11, supplementary FIG. 5).

Next, the present inventors evaluated the effect of CiRS-7 expression upon miR-7-dependent regulation of endogenous targets.

To this end, a single copy of the CiRS-7 expression vector was stably inserted into Flp-In HeLa cells that otherwise have no or very little endogenous expression of CiRS-7.

Analysing the expression levels of the constructed HeLa cells by northern and qRT-PCR revealed CiRS-7 levels comparable to the endogenous levels observed in HEK293, however approximately 100-fold less than total brain levels (FIG. 12, supplementary FIG. 6a).

As in HEK293 cells, the stably expressed CiRS-7 in HeLa cells was sensitive towards miR-671 as previously published14 but remained resistant towards miR-7 (FIGS. 3c and 3g; FIG. 12, supplementary FIG. 6b).

Transfecting miR-7 in a dose-gradient manner revealed that established miR-7 targets (SNCA22, EGFR23 and IRS224) responded more efficiently in the empty cell line without CiRS-7 expression (HeLa-EV) compared to CiRS-7-expressing cells (HeLa-CiRS-7) (FIG. 3d-f).

In addition, transfecting miR-671 into HeLa-CiRS-7 prior to introducing dose-gradient miR-7 levels, the evaluated target genes regained miR-7 sensitivity compared to control transfections (FIG. 3h-j).

This suggests that miR-671 likely functions as an indirect regulator of miR-7 activity by targeting and reducing CiRS-7 levels.

The existence of a circular RNA acting as a potent miRNA sponge immediately questions whether circular miRNA sponges are a more general phenomenon.

Indeed, by predicting miRNA target sites within the other well-known, testis-specific circular SRY RNA, the present inventors identified 16 putative target sites for miR-138 (FIG. 4a).

To validate SRY as a miR-138 target transcript, the present inventors inserted the SRY sequence into the 3′UTR of renilla and tested the knockdown potential of miR-138, expressed from a vector (FIG. 13, supplementary FIG. 7a).

Indeed, upon co-expression miR-138 with psiCheck-SRY the present inventors observed a dramatic and significant reduction of renilla luminescence compared to control experiments (FIG. 4b).

Next, the present inventors devised a vector expressing circular SRY RNA (FIG. 13, supplementary FIG. 7b) to study a direct interaction between miR-138 and SRY. Proper biogenesis of circular SRY was verified by northern blot (FIG. 13, supplementary FIG. 7c) and RT-PCR (FIG. 13, supplementary FIG. 7d).

Immunoprecipitation of myc-tagged Agog from miR-138 transfected cells resulted in enrichment of SRY RNA compared to miR-769 transfected cells (FIG. 4c).

Additionally, using biotin-labelled miR-138, SRY RNA was specifically captured (FIG. 4d), suggesting that SRY indeed is able to interact with miR-138 and—like CiRS-7—acts as a miRNA sponge.

Thus the present inventors propose that the circular SRY be denoted CiRS-138.

Our observation that very abundant endogenous circular RNA molecules, which are inherently resistant to exo-nucleolytic RNA-decay, can serve as efficient miRNA sponges, adds to the growing cellular repertoire of regulatory functions in gene expression.

Conclusively, considering the widespread involvement of miR-7 in cancer as a key regulator of various cancer pathways and also the suggested implication of miR-7 in Parkinson Disease by direct targeting of a-synuclein protein expression, CiRS-7 likely serves as a crucial factor significantly engaged in the functioning of neurons as well as a responsible candidate in neurological disorders and brain tumour development.

Moreover, identifying circular SRY as a CiRS-138 potentially sheds light on the early but undisclosed mechanisms governing male determination.

### Materials and Methods

### Constructs and Transfections

HEK293 Flp-In T-Rex cells (Invitrogen) were maintained under standard culture conditions. Transfections were conducted using either calcium phosphate using standard procedures or Lipofectamine 2000 (Invitrogen) according to supplier's protocol. The construction of pJEBB-769 has previously been published.

pJEBB-7 and pJEBB-138 were constructed similarly; PCR amplification of miR-7-1 locus using miR-7 FW/miR-7 RE or miR-138 locus (in mouse) using miR-138 FW/miR-138 RE and inserting the fragments into a NotI and SalI digested eGFP expression cassette (pJEBB).

pCK-7 was generated by digesting pJEBB-7 with NotI and XbaI and inserting the resulting fragment into NotI and XbaI digested pCK.

pcDNA3-CiRS-7-ir was constructed by PCR amplifying the cRNA locus including 1000 bp upstream and 200 bp downstream the non-linear splice sites using CiRS-7 FW/CiRS-7 RE.

The PCR fragment was inserted into HindIII and NotI digested pcDNA3 (Invitrogen).

An approximately 800 bp DNA stretch upstream the SA was amplified using CiRS-7ir FW/CiRS-7ir RE and inserted in the reverse orientation downstream in an XhoI digested pcDNA3-CiRS-7-ir, thus generating the pcDNA3-CiRS-7.

For the generation of pcDNA3-CiRS-fs, PCR amplification of CiRS-7 exon using CiRS-7ir FW/CiRS-7ir RE was inserted into pcDNA3 using HindIII and XhoI.

To establish HeLa cells stably expressing CiRS-7, CiRS-7 was copy-pasted into pcDNA5 using HindIII and Apal and transfected into Flp-In™ HeLa cells (Invitrogen) along with pOG44. 48 hrs after transfections, the cell media was supplemented with hygromycin B according to manufacturer's protocol.

pcDNA3-SRY was constructed by PCR amplifying the SRY locus from male mouse DNA using SRY FW/SRY RE and inserting the amplicon into pcDNA3 using BamHI and NotI. Subsequently, SalI digested CMV promoter from pcDNA5 was inserted into the XhoI-site in an inverse orientation.

Luciferase reporter vectors were constructed by inserting annealed primers (miR-7-psiCheck FW/miR-7-psiCheck RE) or PCR amplicons produced by CiRS-7-psiCheck FW/CiRS-7-psiCheck FW, SRY-psiCheck FW/SRY-psiCheck RE or EGFR-psiCheck FW/EGFR-psiCheck RE into psiCheck-2 vector.

Dose-gradient transfection of miR-7 mimic (Applied Biosystems) was conducted using Lipofectamine 2000 (Invitrogen). In each case, a total concentration of 10 nM was used with varying ratios between miR-7 and N.C (negative control) as indicated.

### Northern Blot

For miRNA, 20-30 μg whole cell RNA was loaded on a 12% denaturing PAGE gels. RNA was transferred to Amersham hybondTM-N+ membranes (GE Healthcare). The membranes were hybridized with 32P-labelled DNA sis (listed in Table S2) in Church buffer (0.5M NaPO4, 7% SDS, 1 mM EDTA, 1% BSA, pH 7.5) at 37° C. and washed in 2×SSC (300 mM NaCl, 30 mM Na-citrate, pH 7.0) with 0.1% SDS at room temperature. The membranes were exposed on phosphorimager screens and analyzed using Bio-Rad Quantity One® software (Bio Rad).

For cRNA, agarose northern blot was performed with 5-10 μg RNA separated in 1.2% agarose. Subsequent hybridization and wash was carried out at 50° C., otherwise conducted as described above.

### Ago-IP

Myc-Tagged Agog was co-transfected with either pJEBB-7 or pJEBB-769 in p10 dishes. Forty eight hours post-transfection, Ago-IP was performed as described previously26. Briefly, cells were lysed in 150 mM KCl, 25 mM Tris-HCl pH 7.4, 5 mM EDTA, 0.5% Triton X-100, 5 mM DTT supplemented with Ribolock (Fermentas) and proteinase inhibitor cocktail (Roche). The lysate was mixed with myc-antibody (Abcam, ab9106) coupled sepharose beads (GE Healthcare) and left under rotation for 4 hrs at 4° C. Beads were subsequently washed 5 times in lysis buffer and the RNA was extracted using TRIzol reagent (Invitrogen).

### Streptavidin Aptamer

T7 transcription was performed on PCR products containing the entire cRNA sequence with or without the inclusion of a streptavidin aptamer sequence using T7 MEGAscript (Ambion). The streptavidin-based capturing of RNA with associated proteins was conducted as previously published20.

Briefly, the T7 transcript was renatured and mixed with streptavidin beads (Dynabeads MyOne™ Streptavidin Cl, Invitrogen) for 1 hr at 4° C. and washed twice times in lysis buffer (10 mM HEPES pH 7.0, 200 mM NaCl, 1% Triton X-100, 10 mM MgCl2 and 1 mM DTT).

Cells transfected 48 hrs prior to harvest with either pJEBB-7 or pJEBB-769 were lysed and added to the RNA coupled beads using half a confluent p10 dish per streptavidin capture. After rotating for 4 hrs at 4° C., the beads were washed 5 times and RNA and protein were extracted and analysed by northern blot with probes listed in Table S2 and by western blot using anti-Ago2 (Abcam, ab57113) and anti-HuR (Santa Cruz, SC-5261) as a load control.

Biotin-Coupled miRNA Capture

Biotin-labelled miRNAs were transfected into HEK293 cells at a final concentration of 20 nM. Forty eight hours post transfection, cells were harvested and treated as described above.

### IF and RNA-FISH

Cells were seeded on glass coverslips in 12-well plates and transfected with CiRS-7 and pCK-7 using calcium phosphate (HEK293 cells) or Lipofectamine 2000 (HeLa cells). 48 hrs post-transfection, cells were washed in PBS and fixed in 4% paraformaldehyde for 15 min and permeabilized overnight in 70% ethanol. Cells were then washed twice in PBSM, and rehydrated for 10 min in 50% formamide and 2×SSC.

In case of IF, cells were blocked with 3% BSA in PBSM for 1 hr followed by incubation with primary antibody (hDcp1A27) in 3% BSA/PBSM supplemented with RNaseOUT (Fermentas) at 37° C. After washing three times in PBSM, cells were incubated with secondary antibody (Alexa Flour 488, A21206, Invitrogen). Then, cells were washed 3 times before proceeding with RNA FISH.

In case of FISH, cells were incubated at 37° C. in a solution containing 50% formamide, 2×SSC, 0.25 mg/mL Escherichia coli tRNA, 0.25 mg/mL salmon sperm DNA (Invitrogen), 2.5 mg/mL BSA (Roche), and 0.5 ng/mL fluorescently labelled CiRS-7 (FIG. 6, table S2) and miR-7 (Exiqon, 38485-02) probes. After 3 hrs, cells were washed twice for 20 min at 37° C. in 50% formamide and 2×SSC, followed by four 5-min washes in PBS (the penultimate wash containing DAPI) and an additional brief wash in nuclease-free water. Cells were mounted in ProLong Gold (Invitrogen) and left overnight at room temperature.

### Mice for the In Vivo Study.

This study was performed in accordance with guidelines approved by the Danish Animal Ethical Committee (J. no. 2011/561-1950), using 8-9 week old C57BL/6 male mice (n=5) (Taconic, Denmark). The mice were euthanized by cervical dislocation, the brains frozen in gaseous CO2 and processed into 30 μm coronal cryostat sections.

### In Situ Hybridization

In situ hybridization was performed as previously described using a custom designed alkaline phosphatase-labelled DNA and LNA probes (FIG. 6, table S2) recognizing GAPDH, CiRS-7 and miR7, respectively (DNA Technology A/S, Denmark). The specificity of the signal was tested by hybridizing parallel sections 1) with 100-fold excess of unlabeled probe, 2) pre-treatment with RNase A (Pharmacia Biotech), or 3) buffer alone. All controls were devoid of signal.

### Subcellular Fractionation

Subcellular fractionation was performed as previously described. Briefly, cells grown in p10-dishes were gently lysed in 200 ul lysis buffer (10 mM Tris, pH 8.0, 140 mM NaCl, 1.5 mM MgCl2, 0.5% Nonidet P-40), incubated on ice, and centrifuged for 3 min at 1000×g. The supernatant was kept as the cytoplasmic fraction. The nuclear pellet was washed three times in 1 mL lysis buffer. RNA was subsequently purified using TRIzol (Invitrogen).

### Luciferase Reporter Assay

HEK293 cells seeded in 12-well plates were transfected with 0.5 μg pcDNA3/pcDNA3-CiRS-7, 0.2 μg pJEBB-7/pJEBB-769, and 0.1 μg psiCheck vector, and harvested after 48 hrs using the Dual-Luciferase Reporter Assay kit (Promega). In case of anti-miR experiments, anti-miR™ miRNA Inhibitor (MH10047, Applied Biosystems) was used at a final concentration of 50 nM. Luminescence was measured on a BMG FLUOstar luminometer (BMG labtech, Offenburg Germany).

RT-PCR and qPCR

Total RNA was extracted from cells in culture using TRIzol reagent (Invitrogen) according to standard procedures. cDNA for qRT-PCR expression analyses was synthesized from total RNA by MLV-RT (Invitrogen) according to the supplied protocol using random hexamer primer. qPCR was performed with Platinum SYBRGreen qPCR Supermix UDG (Invitrogen) on either a MxPro3000 cycler (Stratagene) or on a LightCycler 480 (Roche) according to standard procedures using primers listed in FIG. 6, Table S2. RNA levels were normalized to GAPDH.

In case of miRNA taqman qPCR, miR-7 (000386) and miR-769 (001998) assays (Applied Biosystems) were used adhering to manufacturer's protocol.

### Assay I

A preferred target sequence in the circular RNA has at least one mismatch to the microRNA that make it resistant to endonucleolytic cleavage. A preferred sequence is resistant to endonucleolytic cleavage by the microRNA it regulate as interpreted from a simple assay where both the circular RNA and microRNA is expressed in the same cell (See FIG. 12b). The assay, here demonstrated for microRNA-7, can be conducted by a skilled scientist for any other part of circular RNA and microRNA. As an example of a micro-RNA that do not contain any mismatches in the central part, and therefore is able to cleave the circular RNA, microRNA-671 included.

The assay is a Northern blot using RNA from HeLa cells with empty vector expression cassette as a control for probe specificity (HeLa-EV, FIG. 12b) or Hela cells expressing circular RNA with 77 target sites for microRNA-7 (HeLa-ciRS-7, FIG. 12b). The blot was probed with antisense oligo complementary to the circular RNA (upper panel) and 18S (lower panel, loading control). The asterix (*) denotes the circular RNA.

The Hela cells were transfected with 10 nM non-related control microRNA (N.C.), microRNA-7 or miR-671 mimics (Applied Biosystems) as indicated using Lipofectamine 2000 (Invitrogen). RNA was harvested 48 hours post transfection using TRIzol reagent (Invitrogen). The northern blot was performed with 10 ug RNA separated in 1.2% agarose. RNA was transferred to Amersham hybond-N+ membrane (GE Healthcare). The membrane was hybridized with [32P]-labelled DNA oligonucleotides (ciRS-7 probe: TTGGAAGACTTGAAGTCGCTGGAAGACCCGGAGTTGTTGGAAGACCTTGACACAGGTGC C, 18S probe: TTACCGCGGCTGCTGGCACCAGACTTGCCCTCCAATGGATCCTCGTTAAAGGATTTAAAG TGGACTCATTCCAATTACAG) in Church buffer (0.5M NaPO4, 7% SDS, 1 mM EDTA, 1% BSA, pH7.5) at 55° C. and washed three times in 2×SSC (300 mM NaCl, 30 mM Na-citrate, pH7.0) with 0.1% SDS at 50° C. The membrane was exposed on phosphorimager screen and analysed using Quantity One or Image Lab software (Bio Rad).

### Assay II

A preferred sponge sequence in the circular RNA has at least one mismatch to the microRNA that makes it resistant to Agog-mediated endonucleolytic cleavage. A preferred sponge sequence is resistant to endonucleolytic cleavage by the microRNA, thus instead of being cleaved by the microRNA, it sequesters and inhibits the microRNA in cells where both the circular RNA and microRNA is expressed (See FIG. 14). The assay, here demonstrated for microRNA-7, can be conducted by a skilled scientist for any other pair of circular RNA and microRNA. The circular RNA in this assay contains 74 sponge sites for microRNA-7 and 1 cleavage site for microRNA-671. Therefore, the observed effect is decreased and unchanged levels of circular RNA when introduced to microRNA-671 and microRNA-7, respectively (FIG. 14).

Total RNA was extracted from cells in culture using TRIzol reagent (Invitrogen) according to standard procedures. Complementary DNA for qRT-PCR expression analyses was synthesized from 1 ug total RNA by MLV-RT (Invitrogen) according to the supplied protocol using random hexamer primer. Quantitative PCR was performed with Platinum SYBR Green qPCR Supermix UDG (Invitrogen) on either MxPro3000 cycler (Stratagene) or LightCycler 480 (Roche), using primers specific for ciRS-7 (ACGTCTCCAGTGTGCTGA/CTTGACACAGGTGCCATC) or GAPDH (GTCAGCCGCATCTTCTTTTG/GCGCCCAATACGACCAAATC).

1. An isolated circular RNA polynucleotide, which is at least partly complementary to a human or animal microRNA sequence.

2-40. (canceled)

41. The circular RNA polynucleotide according to claim 1, having at least one mismatch to said microRNA.

42. The circular RNA polynucleotide according to claim 1, wherein said circular RNA polynucleotide is resistant to endonucleolytic cleavage caused by said microRNA.

43. The circular RNA polynucleotide according to claim 1, wherein said circular RNA polynucleotide contains more than one binding site for microRNA.

44. The circular RNA polynucleotide according to claim 1, wherein said circular RNA polynucleotide comprises at least two, three, four, five, six, seven, eight, or nine binding sites for microRNA.

45. The circular RNA polynucleotide according to claim 1, wherein said circular RNA polynucleotide comprises at least ten, fifteen, twenty, twenty five, thirty, or thirty five binding sites for microRNA.

46. The circular RNA polynucleotide according to claim 1, wherein said circular RNA polynucleotide is present in a medicament.

47. The circular RNA polynucleotide according to claim 1, wherein said circular RNA polynucleotide is present in an in vivo or in vitro in laboratory experiment.

48. The circular RNA polynucleotide according to claim 47, wherein said in vivo or in vitro laboratory experiment comprises an inhibition of miR-15, miR-16, miR-17, in cell cultures or organisms.

49. The circular RNA polynucleotide according to claim 1, wherein said circular RNA polynucleotide comprises a splice acceptor site (SA) and a splice donor site (SD).

50. The circular RNA polynucleotide according to claim 1, wherein said circular RNA polynucleotide comprises a region of contiguous nucleobase sequence, which is 100% complementary to the human or animal microRNA seed region.

51. The circular RNA polynucleotide according to claim 1, wherein said circular RNA polynucleotide comprises a region of contiguous nucleobase sequence, which is partially complementary to the human or animal microRNA seed region.

52. The circular RNA polynucleotide according to claim 1, wherein at least part of said circular RNA polynucleotide is complementary to the mature human or animal microRNA sequence.

53. The circular RNA polynucleotide according to claim 1, comprising an antisense sequence that comprises a guide sequence corresponding to the seed sequence of microRNA.

54. The RNA polynucleotide according to claim 1, wherein at least one of the nucleobase units of said circular RNA polynucleotide is a Locked Nucleic Acid (LNA) unit, a Unlocked Nucleic Acid (UNA) unit, a TINA unit, a 2′-OMe RNA unit or a 2′-fluoro RNA unit.

55. A cell comprising the circular RNA polynucleotide according to claim 1.

56. A pharmaceutical composition comprising the circular polynucleotide according to claim 1, and at least one of a pharmaceutically acceptable diluent, carrier and adjuvant, wherein said circular RNA polynucleotide is at least partly complementary to a human or animal microRNA sequence or comprises a guide sequence complementary to the seed sequence of a human or animal microRNA sequence.

57. A method of using the circular RNA polynucleotide set forth in claim 1, for the treatment, amelioration or prevention of a disease or medical disorder associated with the presence or over-expression of microRNA.

58. The method according to claim 57, wherein the disease or medical disorder is selected from the group consisting of: Parkinson's disease, cancer, a viral infection, Hepatitis C, diabetes, epilepsy, a neurological disease, insulin deficiency and blood sugar, and the following diseases and medical disorders: Abdominal Aortic Aneurysm, acute lymphoblastic leukemia (ALL), acute myeloid leukemia (AML), acute myocardial infarction, acute promyelocytic leukemia (APL), adenoma, adrenocortical carcinoma, alcoholic liver disease (ALD), Alzheimer's disease, anaplastic thyroid carcinoma (ATC), anxiety disorder, asthma, Astrocytoma, Atopic Dermatitis, autism spectrum disorder (ASD), B-cell chronic lymphocytic leukemia, B-cell lymphoma, Becker muscular dystrophy (BMD), bladder cancer, Brain Neoplasm, breast cancer, Burkitt lymphoma, cardiac hypertrophy, cardiomyopathy, Cardiovascular Disease, Cerebellar neurodegeneration, cervical cancer, cholangiocarcinoma, cholesteatoma, choriocarcinoma, chronic lymphocytic leukemia (CLL), chronic myeloid leukemia (CML), chronic pancreatitis, Colon Carcinoma, colorectal cancer, congenital heart disease, coronary artery disease, Cowden Syndrome, dermatomyositis (DM), Diabetic Nephropathy, diarrhea predominant irritable bowel syndrome (IBS-D), diffuse large B-cell lymphoma (DLBCL), Dilated Cardiomyopathy, Down syndrome (DS), Duchenne muscular dystrophy (DMD), endometrial cancer, Endometrial Endometrioid Adenocarcinoma, Endometriosis, epithelial ovarian cancer (EOC), esophageal cancer, Esophagus squamous cell carcinoma, essential thrombocythemia (ET), facioscapulohumeral muscular dystrophy (FSHD), follicular lymphoma (FL), follicular thyroid carcinoma (FTC), frontotemporal dementia, gastric cancer (stomach cancer), glioblastoma, glioblastoma multiforme (GBM), glioma, glomerular disease, Glomerulosclerosis, hamartoma, HBV-related cirrhosis, HCV infection, head and neck cancer, head and neck squamous cell carcinoma (HNSCC), hearing loss, Heart Disease, heart failure, Hepatitis B, Hepatitis C, hepatocellular carcinoma (HCC), Hilar Cholangiocarcinoma, Hodgkin's lymphoma, homozygous sickle cell disease (HbSS), Huntington's disease (HD), Hypertension, Hypopharyngeal cancer, Inclusion body myositis (IBM), Insulinoma, Intrahepatic cholangiocarcinoma (ICC), kidney cancer, Kidney Disease, laryngeal carcinoma, Late Insomnia (sleep disease), Leiomyoma of lung, Leukemia, limb-girdle muscular dystrophies types 2A (LGMD2A), lipoma, Lung Adenocarcinoma, lung cancer, lymphoproliferative disease, malignant lymphoma, malignant melanoma, Malignant mesothelioma (MM), mantle cell lymphoma (MCL), medulloblastoma, melanoma, meningioma, metabolic disease, miyoshi myopathy (MM), multiple myeloma (MM), multiple sclerosis, MYC-rearranged lymphoma, Myelodysplastic Syndrome, myeloproliferative disorder, myocardial infarction, myocardial injury, myoma, nasopharyngeal carcinoma (NPC), nemaline myopathy (NM), Nephritis, neuroblastoma (NB), neutrophilia, Niemann-Pick type C (NPC) disease, non-alcoholic fatty liver disease (NAFLD), non-small cell lung cancer (NSCLC), Obesity, Oesophageal adenocarcinoma, Oral Carcinoma, osteosarcoma, ovarian cancer (OC), pancreatic cancer, pancreatic ductal adenocarcinoma (PDAC), Pancreatic Neoplasia, Panic Disease, papillary thyroid carcinoma (PTC), Parkinson's disease, PFV-1 infection, Pharyngeal Disease, pituitary adenoma, Polycystic Kidney Disease, Polycystic liver disease, polycythemia vera (PV), polymyositis (PM), primary biliary cirrhosis (PBC), primary myelofibrosis, prion disease, prostate cancer, Psoriasic Arthritis, psoriasis, pulmonary hypertension, recurrent ovarian cancer, Renal Cell Carcinoma, renal clear cell carcinoma, retinitis pigmentosa (RP), retinoblastoma, rhabdomyosarcoma, Rheumatic Heart Disease and Atrial Fibrillation, Rheumatoid Arthritis, sarcoma, schizophrenia, sepsis, serous ovarian cancer, Sezary syndrome, skin disease, Small Cell Lung Cancer, Spinocerebellar ataxia, squamous carcinoma, T-cell leukemia, teratocarcinoma, testicular germ cell tumor, thalassemia, thyroid cancer, tongue squamous cell carcinoma, tourette's syndrome, type 2 diabetes, ulcerative colitis (UC), uterine leiomyoma (ULM), uveal melanoma, vascular disease, vesicular stomatitis, and Waldenstrom Macroglobulinemia (WM).
